(19) United States
(12) Patent Application Publication
(10) Pub. No.: US 2006/0234956 A1
(43) Pub. Date: Oct. 19, 2006

(54) Dicarboxylic acid ester derivatives of ginsenoside, pharmaceutical preparations containing the same, and preparation thereof

(75) Inventors: Hui-Ling Chen, Miao-Li (TW); Ying-Ming Huang, Miao-Li (TW); Ching-Te Chang, Miao-Li (TW); Wen-Yi Chuang, Miao-Li (TW)

Correspondence Address: JACKSON WALKER LLP, 901 MAIN STREET, SUITE 6000, DALLAS, TX, 75202-3797, US

(73) Assignee: Amersen Bioscience International, Inc., Miao-Li (TW)

(21) Appl. No.: 11/404,303

(22) Filed: Apr. 14, 2006

Related U.S. Application Data
(60) Provisional application No. 60/671,878, filed on Apr. 15, 2005.

(51) Int. Cl. C07J 17/00; C07J 9/00; A61K 31/704; A61K 31/56

(52) U.S. Cl. 514/26; 514/169; 536/6.1; 552/540

(57) ABSTRACT

The present invention relates to a series of dicarboxylic acid ester derivatives of ginsenosides such as succinate and glutarate derivatives of 20-O-β-D-glucopyranosyl-protopanaxadiol (compound K, abbreviated as CK), preparation thereof and pharmaceutical uses thereof. The dicarboxylic acid ester derivatives of ginsenosides of the present invention can be used to form pharmaceutical acceptable salts thereof having a high water solubility or can be directly dissolved in an aqueous solution of metal salt, and retain the pharmaceutical activities of ginsenosides such as tumor growth inhibition and cancer preventive cytotoxicity. The dicarboxylic acid ester derivatives of ginsenosides of the present invention are thus suitable for use in the manufactures of various pharmaceutically and cosmetically acceptable dosage forms of preparations, such as peripheral, oral, and topical dosage forms.

### CROSS-REFERENCE TO RELATED APPLICATION

This application claims the benefit of U.S. Provisional Application No. 60/671,878, filed 15 Apr. 2005.

### FIELD OF THE INVENTION

The present invention relates to a series of dicarboxylic acid ester derivatives of ginsenoside, and applications of using such derivatives as medicine.

### BACKGROUND OF THE INVENTION

Ginseng has many physiological and pharmacological effects, such as anti-cancer, enhancing immunity, regulating blood-glucose level, anti-ageing, enhancing memory and learning capabilities, etc. The pharmacological properties of ginseng are generally attributed to its triterpene glycosides, called ginsenosides. Ginsenosides are mainly dammarane triterpenes with protopanaxadiol (PPD) and protopanaxatriol (PPT) aglycon moieties. According to the molecular structures, ginsenosides mainly can be divided into protopanaxadiols, protopanaxatriols, and oleanolic acid-type saponin, wherein Ra₁, Ra₂, Ra₃, Rb₁, Rb₂, Rb₃, Rc, Rd, F₂, Rg₃, Rg₅, Rh₂, Rh₃, Rk₁, etc. belong to protopanaxadiols, Re, Rg₁, Rg₂, Rg₄, Rh₁, Rh₄, etc. belong to protopanaxatriols, and Ro belongs to oleanolic acid-type saponin. Ra, Rb, Rc, Rd, and Re, etc. are ginsenosides containing at least 3˜5 sugar moieties which are relatively more abundant in white ginseng have relatively better water solubility. On the other hand, other ginsenosides containing only 1˜2 sugar moieties (e.g. Rg₃, Rg₅, Rh₁, Rh₂, Rh₃, Rk₁, etc.) or even free of sugar moiety (PPD, PPT, etc.) which are rare ginsenosides present only in heat treated red Panax ginseng and have stronger physiological activities, such as anti-cancer effect, but are more hydrophobic.

Ginsenoside Compound K (CK) is an important ingredient with a concentration of only a few tens of ppm in Panax ginseng. Many studies have indicated that Ginsenoside CK has excellent clinical therapeutic and preventive effects on malignant tumors. U.S. Pat. No. 5,919,770 and its parallel patents CN patent 1,182,433A and WO 97-31013 have disclosed the metabolites of ginseng saponins by human intestinal bacteria and the preparation of an anticancer agent. One of the main metabolites is CK, and the preparation from said disclosure is a potential anti-cancer agent with immunity enhancement function and capable of inhibiting the vascularization of tumors and extravasation of cancer cells. CN Patent 1,417,345A and its parallel patent WO03-04383 have disclosed a method for preparing CK by enzyme hydrolysis of ginseng saponins, which is for preparing anti-cancer medicine.

US patent publication 2002-00132260 A and its parallel CN patent 1415304 A and CN patent 1225366 A disclose the use of ginsenoside Rh2 in pharamaceutical compositions used in methods of inhibiting the multiplication of cancer cells, and a method for preparing Rh2. CN patent 1417225 A discloses a method of preparing a ginsenoside Rg₃ by acid hydrolysis, which is for a preparation of anti-tumor medicine. US patent publication No. 2003-0092638 A and its parallel WO patent WO03-024459 disclose a use of ginsenosides PPD and PPT as an anti-cancer helper. CN patent 1418633 A discloses an application of ginsenoside PPD as an anti-cancer helper. CN patent 1280845 A discloses an anti-cancer pharmaceutical composition comprising ginsenoside Rh₁ as an effective ingredient. US patent publication 2003-0190378 and its parallel WO patent WO03-086438, and US patent publication 2003-0190377 and its parallel WO patent WO03-086439 disclose methods for preparing rare ginsenosides such as Rk₂, Rh₃, PPD, Rg₃, Rg₅, and Rk₁; and methods of using them in treating and preventing cancers or allergic diseases.

CK and other ginsenosides with a few carbohydrate moieties have low polarity and poor water solubility. Dissolution enhancers (e.g. Cremophor emulsifier (polyoxyethylated castor oil), dimethylsulfoxide (DMSO), ethanol, etc.) are required for the pharmaceutical and cosmetic preparation. The toxicity of these dissolution enhancers often restricts the utilities of these ginsenosides. Thus, the present invention plans to perform a series of systematic chemical modifications on CK and the above-mentioned ginsenosides in order to enhance its water solubility and thus its utilities.

### SUMMARY OF THE INVENTION

One primary objective of the present invention is to provide a dicarboxylic acid ester derivative of ginsenoside, pharmaceutical preparation and cosmetic preparation containing the same, and preparation thereof.

Another objective of the present invention is to provide a derivative of 20-O-β-D-glucopyranosyl-protopanaxadiol (compound K, abbreviated as CK), pharmaceutical preparation and cosmetic preparation containing the same, and preparation thereof.

In order to accomplish the aforesaid objectives, a ginseng saponin or ginsenoside is reacted with an activated dicarboxylic acid derivative,, such as dicarboxylic anhydride, dicarboxylic halide or dicarboxylic ester etc. in a suitable solvent and in the presence of a catalyst to form a dicarboxilyic acid ester, the reaction of which using diacid anhydride can be represented by the following equation: 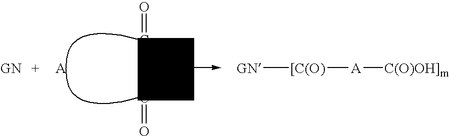
wherein m is a positive integer; GN is a ginsenoside; and GN′ is a residual resulting from m number of hydroxyl groups of GN reacted with the diacid anhydride, i.e. —OH being converted to —O— which is bonded to —C(O)—A—C(O)OH; and A is —(CR⁴R⁹)ₙ—, wherein R⁴ and R⁹ independently are H or C1-C4 alkyl, and n is an integer of 0-8.

The ginsenoside, GN, suitable for use in the present invention includes (but not limited to): 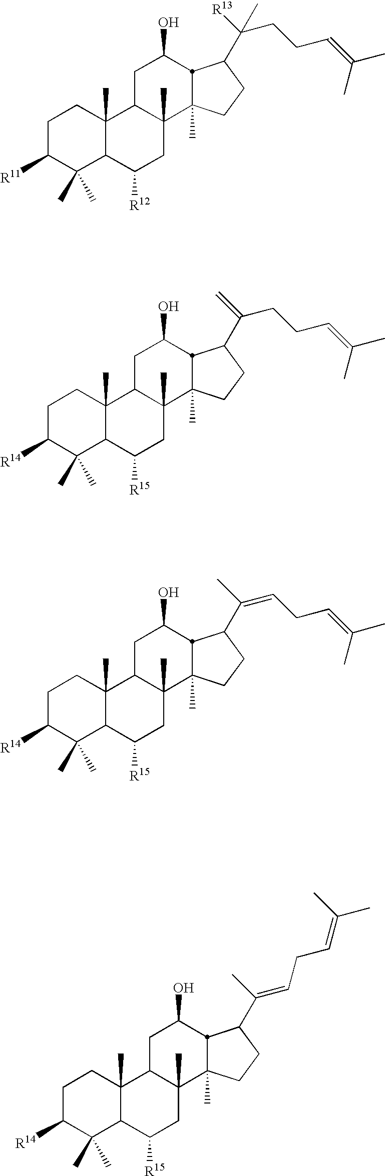
wherein R¹¹ is —OH, —O—Glc or —O—Glc—Glc; R¹² is H, —OH, —O—Glc, —O—Glc—Glc or —O—Glc—Rha; R¹³ is —OH or —O—Glc; R¹⁴ is —OH, —O—Glc, —O—Glc—Glc, or —O—Glc—Glc—Ac; and R¹⁵ is —H, —O—Glc, —O—Glc—Ac, or —O—Glc—Rha, wherein Glc— is 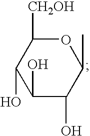
Glc—Glc— is 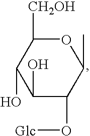
wherein Glc— is defined as above;
Rha—Glc— is 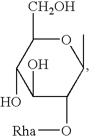
wherein Rha— is 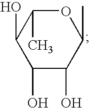
Ac—Glc— is 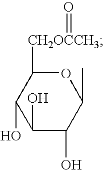
Ac—Glc—Glc— is 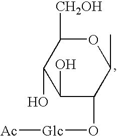
wherein Ac—Glc— is defined as above.

Typical examples of the ginsenosides, GN, are as follows:

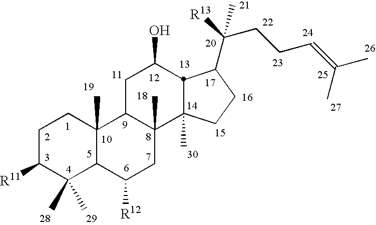

| Ginsenosides | R¹¹ | R¹² | R¹³ |
|---|---|---|---|
| PPD | HO— | H— | HO— |
| Rh₂ | Glc-O— | H— | HO— |
| Rg₃ | Glc-β-1,2-Glc-O— | H— | HO— |
| CK | HO— | H— | Glc-O— |
| F2 | Glc-O— | H— | Glc-O— |
| PPT | HO— | HO— | HO— |
| Rh₁ | HO— | Glc-O— | HO— |
| F1 | HO— | HO— | Glc-O— |
| Rf | HO— | Glc-β-1,2-Glc-O— | HO— |
| Rg₁ | HO— | Glc-O— | Glc-O— |
| Rg₂ | HO— | Rha-α-1,2-Glc-O— | HO— |

| 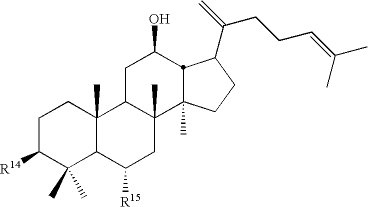 | | | | 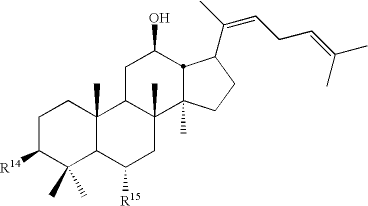 | |
|---|---|---|---|---|---|
| | Ginsenosides | R¹⁴ | R¹⁵ | R¹⁴ | R¹⁵ |
| | Rg₆ | HO— | Rha-Glc-O— | | |
| | Rk₁ | Glc-Glc-O— | H— | | |
| | Rk₂ | Glc-O— | H— | | |
| | Rk₃ | HO— | Glc-O— | | |
| | Rs₅ | Ac-Glc-Glc-O— | H— | | |
| | Rs₇ | HO— | Ac-Glc-O— | | |
| | F4 | | | HO— | Rha-Glc-O— |
| | Rg₅ | | | Glc-Glc-O— | H— |
| | Rh₃ | | | Glc-O— | H— |
| | Rh₄ | | | HO— | Glc-O— |
| | Rs₄ | | | Ac-Glc-Glc-O— | H— |
| | Rs₆ | | | HO— | Ac-Glc-O— |

The dicarboxylic acid ester of ginsenoside synthesized in the present invention comprises at least one group of —OC(O)—A—C(O)OH which replaces —OH of the ginsenoside, wherein A is —CR⁴R⁹)ₙ—, wherein R⁴ and R⁹ independently are H or C1-C4 alkyl, and n is an integer of 0-8, which may be represented (but not limited to) by the following formulas: 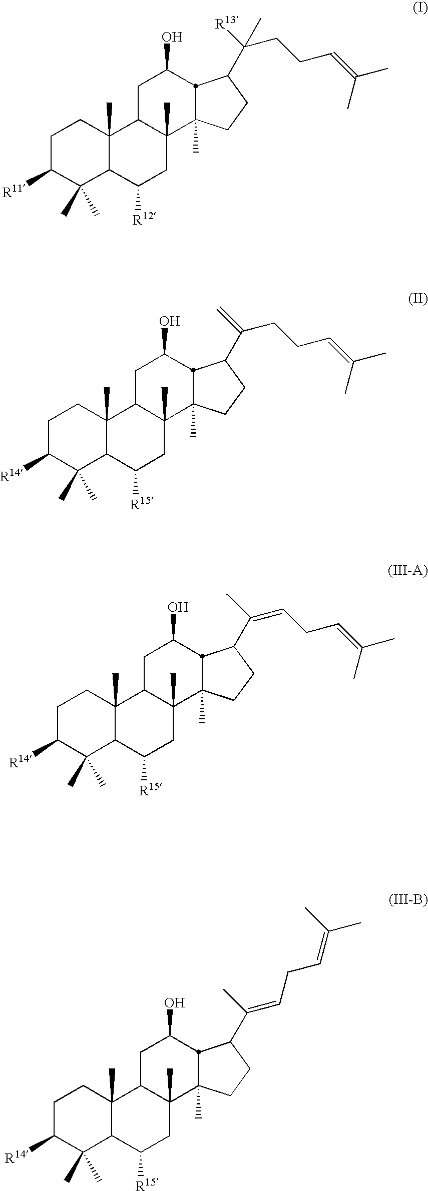
wherein the dicarboxylic acid ester of (I) comprises the following (I′), (I″) or a racemic mixture thereof: 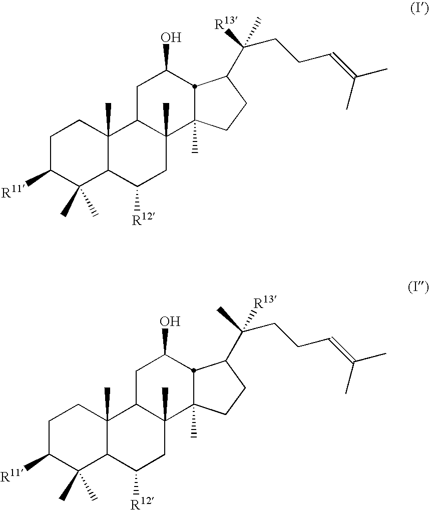
wherein R^{11′} is R¹¹, —OE, —O—Glc′ or —O—Glc—Glc′; R^{12′} is R¹², —OE, —O—Glc′, —O—Glc—Glc′ or —O—Glc—Rha′; R^{13′} is R¹³, —OE or —O—Glc′; R^{14′} is R¹⁴, —OE, —O—Glc′, —O—Glc—Glc′, or —O—Glc—Glc—Ac′; and R^{15′} is R¹⁵, —O—Glc′, —O—Glc—Ac′, or —O—Glc—Rha′, wherein R¹¹, R¹², R¹³, R¹⁴ and R¹⁵ are defined as above; E is —C(O)(CR⁴R⁹)ₙCOOH, wherein R⁴, R⁹ and n are defined as above;

—O—Glc′ is 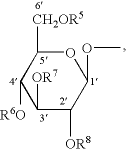
wherein R⁵, R⁶, R⁷ and R⁸ are H or E, wherein E is defined as above;
—O—Glc—Glc′ is 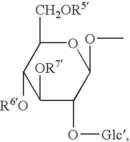
wherein R^{5′}, R^{6′}, and R^{7′} are H or E, wherein E and —O—Glc′ are defined as above; 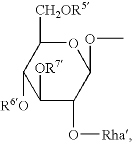
wherein R^{5′}, R^{6′}, and R^{7′} are H or E, wherein E is defined as above, and —O—Rha′ is 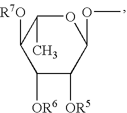
wherein R⁵, R⁶, and R⁷ are defined as above;
—O—Glc—Ac′ is 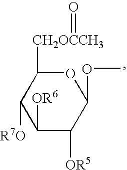
wherein R⁵, R⁶, and R⁷ are defined as above;
—O—Glc—Glc—Ac′ is 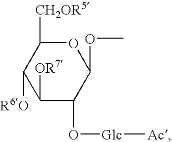
wherein R^{5′}, R^{6′}, R^{7′} and —O—Glc—Ac′ are defined as above.

Preferably, R⁴ and R⁹ are H, and n is 2 or 3.

Preferably, R^{12′} is H, and R^{11′} is —OH, and R_{13′} is —O—Glc′.

Preferably, R^{12′} is H, and R^{11′} is —O—Glc′, and R^{13′} is —OH.

Preferably, R^{12′} is H, and R^{11′} is —O—Glc—Glc′, and R^{13′} is —OH.

Preferably, R^{12′} is H, and R^{11′} is —O—Glc′, and R^{13′} is —O—Glc′.

Preferably, R^{12′} is —O—Glc′, and R^{11′} is —OH, and R^{13′} is —OH.

Preferably, R^{12′} is —O—Glc—Glc′, and R^{11′} is —OH, and R^{13′} is —OH.

Preferably, R^{12′} is —OH′, and R^{11′} is —OH, and R^{13′} is —O—Glc′.

Preferably, R^{12′} is —O—Glc′, and R^{11′} is —OH, and R^{13′} is —O—Glc′.

Preferably, R^{12′} is —O—Glc—Rha′, and R^{11′} is —OH, and R^{13′} is —OH.

Preferably, R^{12′} is —H, and R^{11′} is —OH or —OE, and R^{13′} is —OH or —OE.

Preferably, R^{12′} is —OH or —OE, and R^{11′} is —OH or —OE, and R^{13′} is —OH or —OE.

Preferably, R^{14′} and R^{15′} are one of the following combinations:

| R^{14′} | R^{15′} |
|---|---|
| —OH | —O-Glc-Rha′ |
| —O-Glc-Glc′ | H |
| —O-Glc′ | H |
| —OH | —O-Glc′ |
| —O-Glc-Glc-Ac′ | H |
| —OH | —O-Glc-Ac′ |

The present invention also provides a pharmaceutical preparation, which comprises an aqueous solution, and a dicarboxylic acid ester of ginsenoside of the present invention or a pharmaceutically acceptable salt thereof, dissolved in said aqueous solution. The aqueous solution can be a buffered or non-buffered aqueous solution with or without pharmaceutically acceptable solubility enhancer of said dicarboxylic acid ester of ginsenoside, such as aliphatic alcohol, polyhydroxy alcohol, pharmaceutical or cosmetic acceptable oils, or Cremophor. In the present invention, the terms “pharmaceutical” and “pharmaceutically” also means “cosmetic” and “cosmetically”, respectively. The pharmaceutical preparation may be used as a food supplement or a healthy food, and it has been proved being at least useful in the fabrication of an anti-tumor medicine. A further aspect of the present invention is a method for treating a tumor, the method comprising administering to a subject in need of treatment a dicarboxylic acid ester of ginsenoside of the present invention, or a pharmaceutically acceptable salt thereof, in an amount effective to treat said tumor. A still further aspect of the present invention is the use of a dicarboxylic acid ester of ginsenoside of the present invention or a pharmaceutically acceptable salt thereof in the preparation of a medicament for treating a tumor. The tumor is, for example, OVCAR-3 (Adenocarcinoma, ovary, human), A549 (Carcinoma, lung, human), HT-29 (Adenocarcinoma, colon, moderately well-differentiated grade II, human) or MCF-7 (Breast adenocarcinoma, pleural effusion, human).

Preferably, the concentration of said dicarboxylic acid ester of ginsenoside or a pharmaceutically acceptable salt thereof is 0.01-100 mg/ml in said aqueous solution. Preferably, the concentration is higher than 1 mg/ml and more preferably higher than 10 mg/ml in said aqueous solution.

### DETAILED DESCRIPTION OF THE INVENTION

According to the preferred embodiments of the present invention, derivatives of several typical ginsenosides such as 20-O-β-D-glucopyranosyl-protopanaxadiol (compound K, abbreviated as CK), PPD, PPT, and F1 are synthesized.

In one of the preferred embodiments CK reacts with a dicarboxilyic acid anhydride, in a suitable solvent and in the presence of a catalyst to form a dicarboxilyic acid ester derivative of CK, the reaction can be represented by the following equation: 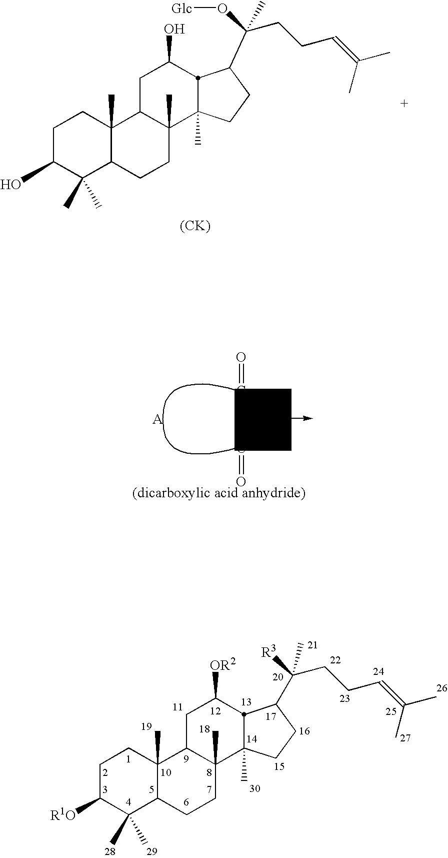
wherein Glc is 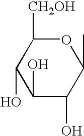
A is —(CR⁴R⁹)ₙ—;
R¹═H or —C(O)(CR⁴R⁹)ₙCOOHH;
R²═H or —C(O)(CR⁴R⁹)ₙCOOH;
R³═ 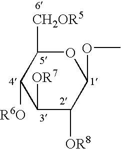
wherein R⁵, R⁶, R⁷ and R⁸ independently are H, or —C(O)(CR⁴R⁹)ₙCOOH;
wherein n is an integer of 0-8, R⁴ and R⁹ independently are H or C1-C4 alkyl, provided that R¹, R², R⁵, R⁶, R⁷ and R⁸ are not H at the same time.

Preferably, R⁴ and R⁹ are H and n is 2 or 3.

Preferably, R² is H.

Preferably, R¹ is H.

Preferably, R⁵ is —C(O)(CH₂)ₙCOOH, and R⁶, R⁷ and R⁸ are all H,.

Preferably, R⁷ is —C(O)(CH₂)ₙCOOH, and R⁵, R⁶ and R⁸ are all H.

Preferably, R⁵ and R⁷ are both —C(O)(CH₂)ₙCOOH, and R⁶ and R⁸ are both H.

Preferably, R⁵, R⁶ and R⁷ are all —C(O)(CH₂)ₙCOOH, and R⁸ is H.

The present invention also provides a pharmaceutical preparation, which includes an aqueous solution and a CK dicarboxylic acid ester derivative of the present invention or a pharmaceutically acceptable salt thereof, which is dissolved in said aqueous solution. The aqueous solution can be a buffered or non-buffered aqueous solution with or without pharmaceutically acceptable solubility enhancer, such as aliphatic alcohol, polyhydroxy alcohol, pharmaceutical or cosmetic acceptable oils, or Cremophor.

Preferably, the concentration of said derivative or a pharmaceutically acceptable salt thereof is 0.01-100 mg/ml in said aqueous solution. Preferably, the concentration is higher than 1 mg/ml and more preferably higher than 10 mg/ml in said aqueous solution.

In the following Examples 1-4, the succinate and glutarate derivatives of CK are synthesized, and their anti-cancer efficacies are compared to that of CK.

The synthesis method comprises mixing CK with succinic anhydride or glutaric anhydride at a mole ratio of 1:0.1˜100, preferably 1:1˜10; and carrying out a ring opening reaction of the dicarboxylic acid anhydride in a suitable solvent and in the presence of a catalyst. Said organic solvent can be halogenated alkane (e.g. dichloromethane, trichloromethane, dichloroethane, etc.; ether (e.g. dialkyl ether or alicyclic ether, tetrahydrofuran, dioxane); tertiary amine including tertiary alkyl amine (e.g. triethylamine, pyridine) and trialkyl diamine (e.g. N,N,N′,N′-tetramethyl ethylenediamine, N,N,N′,N′-tetramethyldiaminomethane); low alkyl sulfoxide (e.g. dimethyl sulfoxide, DMF); and low alkyl ketone. Said catalyst can be a base or an acid. Said base for example can be a tertiary amine, including (but not limited to) trialkyl amine, 4-(dimethylamino)pyridine, pyridine, N,N,N′,N′-tetramethyl ethylenediamine, N,N,N′,N′-tetramethyldiaminomethane, etc.; or solid alkali, such as Si-dimethylamine, Si-morpholine, and Si-piperidine, etc. Preferably, said organic solvent and said catalyst are all tertiary amine. Said ring opening reaction is carried out at −10°˜200° C. (preferably 10˜120° C.) under oscillation or stirring and reflux for 10 minutes to 10 days (preferably 30 minutes to 48 hours). If a water miscible solvent (e.g. tertiary amine or low alkyl sulfoxide) is used in the reaction, the solvent is evaporated first to obtain a concentrate; then partition with water and organic acid ester or ether. The organic layer is concentrated to dry to obtain a product mixture of CK succinate or glutarate. If a solvent immiscible with water is used, the reaction is terminated by directly adding ice water into the reaction solution. Next, the organic layer is obtained and then partition with water and organic acid ester or ether. The organic layer is concentrated to dry to obtain a product mixture of CK succinate or glutarate. Alternatively, a silica gel or reverse phase gel chromatographic column or a high performance liquid phase chromatographic column can be used for further purification of the product mixture.

The CK succinate or glutarate product is dissolved in acetone; followed by dripping a methanol or ethanol solution containing sodium or potassium alcoholate or hydroxide. Upon completion of precipitation, the precipitate is filtered to obtain a potassium or sodium CK succinate salt. A pharmaceutically acceptable salt of said product, e.g. ammonium salt, can also be prepared by a similar method. A CK succinate or glutarate product can also be directly dissolved in a pharmaceutically acceptable saline or an aqueous solution of week base (sodium hydrogen carbonate or potassium hydrogen carbonate) with pH 6˜9 (preferably pH 6.8˜7.8) in order to form a CK succinate or glutarate saline. In case of CK succinate or glutarate salts, they can dissolve in water, or normal saline.

### EXAMPLES

### Example 1

### Synthesis of CK Succinate

401 mg of CK was dissolved in 35 ml of dried THF, and 257 mg (4 equivalents) of succinic anhydride was added to the resulting solution while stirring, to which 393 mg (5 equivalents) of 4-(dimethylamino)pyridine was then added. The reaction nixture was then heated under N₂ at 40° C. for 48 hours. The reaction progress was monitored by reversed-phase TLC, and the solvent was removed by rotary evaporation until dryness, when the reaction was completed. The concentrate was added with ice water and ethyl acetate for partition. The resulting water layer was discarded, and the organic layer was repetitively extracted with water twice. The organic layer was then dried with anhydrous magnesium sulfate and rotary evaporated to dryness to obtain 369.7 mg of CK succinate derivatives mixture product.

The obtained product was analyzed by an analytic HPLC (GL Sciences Inc., Inertsil ODS-3V C-18; 3.0 mm×150 mm) where the mobile phase was 52% acetonitrile/H₂O (containing 0.02% phosphoric acid). The elution times of the main products separately were: 11.0 min (yield: 21.1%), 19.9 min (yield: 44.2%), 24.7 min (yield: 20.7%), 31.7 min (yield: 10.4%). A semi-preparative HPLC (GL Sciences Inc., Inertsil ODS-3 C-18; 10 mm×250 mm) was used for purification separation, thereby obtaining 41.8 mg of 19.9 min product (GS-8), which was identified by mass spectrum (MS) as a CK monosuccinate derivative with a molecular weight of 723.44. Verifications from ¹³C-NMR, ¹H-NMR, H-H COSY, C-H COSY indicated that succinate substituted the hydrogen of the third OH group (3′) on the glucose group at 20^{th} of CK. Furthermore, 14.8 mg of 24.7 min product (GS-9) was obtained, which was identified by MS as a CK disuccinate derivative with a molecular weight of 823.44. Verifications from ¹³C-NMR, ¹H-NMR and H-H COSY indicated that the two succinate groups substituted the hydrogens at the 3^{rd} and 6^{th} OH groups (3′,6′) on the glucose group at 20^{th} of CK. Still furtermore, 11.2 mg of 31.7 min product (GS-11) was obtained, which was identified by MS as a CK trisuccinate derivative with a molecular weight of 923.46. Verifications from ¹³C-NMR, ¹H-NMR and H-H COSY indicated that the three succinate groups substituted the hydrogens at the 3^{rd}, 4^{th} and 6^{th} OH groups (3′,4′,6′) on the glucose group at 20^{th} of CK.

### Example 2

### Synthesis of CK Succinate

41 mg of CK and 40 mg (6 equivalents) of succinic anhydride were dissolved in 40 ml of dried Et₃N. The resulting reaction mixture was then heated under N₂ at 25° C. for 13 hours. The solvent was removed by rotary evaporation until dryness, when the reaction was completed. The concentrate was added with ice water and ethyl acetate for partition for at least three times. The organic layer was then dried with anhydrous magnesium sulfate and rotary evaporated to dryness to obtain 65.3 mg of a mixture product of CK succinate derivatives.

The obtained product was analyzed by a HPLC. The elution times of the main products separately were: 11.0 min (yield: 27.6%), 13.5 min (yield: 41.2%), 19.9 min (yield: 14.4%), 31.7 min (yield: 2.4%). A semi-preparative HPLC was used for purification separation, thereby obtaining 17.5 mg of 13.5 min product (GS-6), which was identified by MS as a CK mono-succinate derivative with a molecular weight of 723.44. Verifications from ¹³C-NMR, ¹H-NMR, and H-H COSY indicated that succinate substituted the hydrogen of the sixth OH group (6′) on the glucose group at 20^{th} of CK.

### Example 3

### Synthesis of CK Glutarate

100 mg of CK was dissolved in 30 ml of dried THF, and 184 mg (10 equivalents) of glutaric anhydride was added to the resulting solution while stirring, to which 158 mg (8 equivalents) of 4-(dimethylamino)pyridine was then added. The reaction mixture was then heated under N₂ at 60° C. for 23 hours. The reaction progress was monitored by reversed-phase TLC, and the solvent was removed by rotary evaporation until dryness, when the reaction was completed. The concentrate was added with ice water and ethyl acetate for partition for at least three times. The organic layer was then dried with anhydrous magnesium sulfate and rotary evaporated to dryness to obtain 135.6 mg of CK glutarate derivatives mixture product.

The obtained product was analyzed by a HPLC. The elution times of the main products separately were: 13.5 min (yield: 3.4%), 20.6 min (yield: 16.0%), 25.3 min (yield: 37.1%), 31.3 min (yield: 3.7%), 36.4 min (yield: 12.5%), and 51.4 min (yield: 25.8%). A semi-preparative HPLC was used for purification separation, thereby obtaining 9.6 mg of 20.6 min product (GS-1 3), which was identified by MS as a CK monoglutarate derivative with a molecular weight of 737.55. Verifications from ¹³C-NMR, ¹H-NMR, H-H COSY, and C-H COSY indicated that glutarate substituted the hydrogen of the 6^{th} OH group (6′) on the glucose group at 20^{th} of CK. Furthermore, 18.1 mg of 25.3 min product (GS-14) was obtained, which was identified by MS as a CK diglutarate derivative with a molecular weight of 851.59. Verifications from ¹³C-NMR, ¹H-NMR and H-H COSY indicated that the two glutarate groups substituted the hydrogen of the 3^{rd} and 6^{th} OH groups (3′,6′) on the glucose group at 20^{th} of CK. Still furthermore, 7.5 mg of 36.4 min product (GS-15) was obtained, which was identified by MS as a CK triglutarate derivative with a molecular weight of 965.64. Verifications from ¹³C-NMR, ¹H-NMR and H-H COSY indicated that the three glutarate groups substituted the hydrogen at the 3^{rd}, 4^{th} and 6^{th} OH groups (3′,4′,6′) on the glucose group at 20^{th} of CK. Still furthermore, 15.5 mg of 51.4 min product (GS-16) was obtained, which was identified by MS as a CK pentaglutarate derivative with a molecular weight of 1193.74. Verifications from ¹³C-NMR, ¹H-NMR and H-H COSY indicated that the five glutarate groups substituted the hydrogen of the OH group at 3^{rd} position of CK, and at the 2^{nd}, 3^{rd}, 4^{th} and 6^{th} OH groups (2!,3′,4′,6′) on the glucose group at 20^{th} of CK.

### Example 4

### Synthesis of CK Glutarate

194 mg of CK and 178 mg (5 equivalents) of glutaric anhydride were dissolved in 150 ml of dried Et₃N. The reaction mixture was then heated under N₂ at 25° C. for 6 hours. The reaction progress was monitored by reversed-phase TLC, and the solvent was removed by rotary evaporation until dryness, when the reaction was completed. The concentrate was added with ice water and ethyl acetate for partition for at least three times. The organic layer was then dried with anhydrous magnesium sulfate and rotary evaporated to dryness to obtain 198 mg of CK glutarate derivatives mixture product.

The obtained product was analyzed by a HPLC. The elution times of the main products separately were: 10.8 min (yield: 31.0%), 13.8 min (yield: 24.6%), 19.4 min (yield: 22.%), 24.3 min (yield: 14.6%), and 31.0 min (yield: 5.2%). A semi-preparative HPLC was used for purification separation, thereby obtaining 34.5 mg of 10.8-min product (GS-12, CK), 30.9 mg of 13.8 min product (GS-17), 23.7 mg of 19.4 min product (GS-13), 23.2 mg of 24.3 min product (GS-14), and 14.4 mg of 31.0 min product (GS-15). GS-17 was identified by MS as a CK monoglutarate derivative with a molecular weight of 737.55. Verifications from ¹³C-NMR, ¹H-NMR, H-H COSY and C-H COSY indicated that glutarate substituted the hydrogen of the 6^{th} OH group (6′) on the glucose group at 20^{th} of CK. Furthermore, were obtained.

CK: ¹H-NMR (500 MHz, CD₃OD) δ: 5.101 (t, 1H, J=5.0 Hz, H-24), 4.600 (d, 1H, J=8.2, H-1′ of 20-Glc), 3.766 (dd, 2H, J=12.5, 2.0 Hz, H-6′a of 20-Glc), 3.672 (td, 1H, J=9.5, 5.0 Hz, H-12), 3.625 (dd, 2H, J=12.5, 5.0 Hz, H-6′b of 20-Glc), 3.346 (td, 1H, J=8.2, 2.0 Hz, H-3′ of 20-Glc), 3.210 (m, 1H, H-5′ of 20-Glc), 3.29 (m,1H, H-4′ of 20-Glc), 3.138 (dd, 1H, J=12.5, 4.5 Hz, H-3), 3.072 (t, 1H, J=8.2 Hz, H-2′ of 20-Glc), 2.283 (td, 1H, J=11.0, 8.5 Hz, H-17), 2.06 (m, 2H, H-23a,b), 1.822 (dd, 2H, J=13.0, 5.0 Hz, H-11α), 1.814 (dt, 2H, J=18.5, 7.5 Hz, H-22a), 1.743 (t, 1H, J=11.0 Hz, H-13), 1.676 (s, 3H, H-26), 1.638 (s, 3H, H-27), 1.39 (m, 2H, H-22b), 1.338 (s, 3H, H-21), 1.220 (dt, 2H, J=13.0, 9.5 Hz, H-11β), 1.017 (s, 3H, H-29), 0.960 (s, 3H, H-28), 0.924 (s, 3H, H-30), 0.914 (s, 3H, H-18), 0.775 (s, 3H, H-19). ¹³C-NMR (125 MHz, CD₃OD) δ: aglycone moiety: C-1, 40.24; C-2, 27.99; C-3, 79.52; C-4, 40.03; C-5, 57.25; C-6, 19.41; C-7, 35.84; C-8, 40.96; C-9, 51.05; C-10, 38.17; C-11, 30.99; C-12, 71.93; C-13, 49.76; C-14, 52.49; C-15, 31.64; C-16, 27.19; C-17, 53.14, C-18, 16.23; C-19, 16.72; C-20, 84.94; C-21, 22.85; C-22, 36.65; C-23, 24.26; C-24, 125.84; C-25, 132.30; C-26, 25.89; C-27, 17.95; C-28, 28.61;-C-29, 16.13; C-30, 17.17; 20-Glc sugar moiety: C-1′, 98.30; C-2′, 75.40; C-3′, 78.19; C-4′, 71.17; C-5′, 77.95; C-6′, 62.50.

CK-6′-monosuccinate (GS-6): ¹H-NMR (500 MHz, CD₃OD) δ: 5.118 (t, 1H, J=6.5 Hz, H-24), 4.582 (d, 1H, J=8.3, H-1′ of 20-Glc), 4.438 (d, 2H, J=11.4 Hz, H-6′a of 20-Glc), 4.104 (dd, 2H, J=11.4, 7.4 Hz, H-6′b of 20-Glc), 3.704 (td, 1H, J=10.5, 4.5 Hz, H-1 2), 3.427 (t,1H, J=8.3 Hz, H-4′ of 20-Glc), 3.344 (dd, 1H, J=8.3, 7.4 Hz, H-5′ of 20-Glc), 3.234 (t, 1H, J=8.3 Hz, H-3′ of 20-Glc), 3.133 (dd, 1.H, J=9.5, 4.5 Hz, H-3), 3.112.(t, 1H, J=8.3 Hz, H-2′ of 20-Glc), 2.279 (td, 1H, J=11.0, 8.5 Hz, H-17), 2.132 (dq, 2H, J=19, 6.5 Hz, H-23a), 2.013 (dq, 2H, J=19, 6.5 Hz, H-23b), 1.891 (dt, 2H, J=18, 6.5 Hz, H-22a), 1.783 (dd, 2H, J=13.3, 4.5 Hz, H-11α), 1.738 (t, 1H, J=11.0 Hz, H-13), 1.671 (s, 3H, H-26), 1.611 (s, 3H, H-27), 1.345 (s, 3H, H-21), 1.243 (dt, 2H, J=13.3, 10.5 Hz, H-11β), 1.004 (s, 3H, H-29), 0.956(s, 3H, H-28), 0.915(s, 3H, H-30), 0.907(s, 3H, H-18), 0.770(s, 3H,H-19).

CK-3′-monosuccinate (GS-8): ¹H-NMR (500 MHz, CD₃OD) δ: 5.109 (t, 1H, J=7.0 Hz, H-24), 4.960 (t, 1H, J=9.5 Hz, H-3′ of 20-Glc), 4.71 (d, 1H, J=8.3, H-1′ of 20-Glc), 3.776 (dd, 2H, J=12.5, 1.8 Hz, H-6′a of 20-Glc), 3.673 (td, 1H, J=11.0, 5.0 Hz, H-12), 3.649 (dd, 2H, J=12.5, 5.0 Hz, H-6′b of 20-Glc), 3.484 (t,1H, J=9.5 Hz, H-4′ of 20-Glc), 3.29 (m, 1H, H-5′ of 20-Glc), 3.215 (dd, 1H, J=9.5, 7.8 Hz, H-2′ of 20-Glc), 3.136 (dd, 1H, J=11.5, 5.0 Hz, H-3), 2.272 (td, 1H, J=11.0, 8.0 Hz, H-17), 2.08 (m, 2H, H-23ab), 1.913 (ddd, 2H, J=19, 13.5, 9.5 Hz, H-22a), 1.828 (dd, 2H, J=13.0, 5.0 Hz, H-11α), 1.737 (t, 1H, J=11.0 Hz, H-13), 1.679 (s, 3H, H-26), 1.622 (s, 3H, H-27), 1.345 (s, 3H, H-21), 1.217 (dt, 2H, J=13.5, 11.0 Hz, H-11β), 1.012 (s, 3H, H-29), 0.958 (s, 3H, H-28), 0.916 (s, 3H, H-30), 0.910 (s, 3H, H-18), 0.771 (s, 3H, H-19).

CK-3′,6,-disuccinate (GS-9): ¹H-NMR (500 MHz, CD₃OD) δ: 5.120 (t, 1H, J=7.3 Hz, H-24), 4.960 (t, 1H, J=9.5 Hz, H-3′ of 20-Glc), 4.685 (d, 1H, J=8.5, H-1′ of 20-Glc), 4.437 (dd, 2H, J=11.5, 2.0 Hz, H-6′a of 20-Glc), 4.139 (dd, 2H, J=11.5, 6.5 Hz, H-6′b of 20-Glc), 3.706 (td, 1H, J=11.0, 5.5 Hz, H-12), 3.537 (ddd, 1H, J=9.5, 6.5, 2.0 Hz, H-5′ of 20-Glc), 3.418 (t,1H, J=9.5 Hz, H-4′ of 20-Glc), 3.260 (dd, 1H, J=9.5, 8.5 Hz, H-2′ of 20-Glc), 3.129 (dd, 1H, J=10.3, 4.8 Hz, H-3), 2.274 (td, 1H, J=10.5, 8.5 Hz, H-17), 2.132 (dq, 2H, J=19.5, 6.8 Hz, H-23a), 2.007 (dq, 2H, J=19.5, 6.8 Hz, H-23b), 1.89 (m, 2H, H-22a), 1.787 (dd, 2H, J=12.8, 5.5 Hz, H-11α), 1.727 (t, 1H, J=11.0 Hz, H-13), 1.673 (s, 3H, H-26), 1.614 (s, 3H, H-27), 1.352 (s, 3H, H-21), 1.211 (dt, 2H, J=12.8, 11.0 Hz, H-11β), 0.999 (s, 3H, H-29), 0.954 (s, 3H, H-28), 0.906 (s, 3H, H-30), 0.906 (s, 3H, H-18), 0.769 (s, 3H, H-19).

CK-3′,4′,6′-trisuccinate (GS-11): ¹H-NMR (500 MHz, CD₃OD) δ: 5.162 (t, 1H, J=9.5 Hz, H-3′ of 20-Glc), 4.914 (t,1H, J=9.5 Hz, H-4′ of 20-Glc), 4.769 (d, 1H, J=7.5, H-1′ of 20-Glc), 4.176 (dd, 2H, J=12.0, 2.0 Hz, H-6′a of 20-Glc), 4.124 (dd, 2H, J=12.0, 6.0 Hz, H-6′b of 20-Glc), 3.793 (ddd, 1H, J=9.5, 6.0, 2.0 Hz, H-5′ of 20-Glc), 3.716 (td, 1H, J=11.0, 5.5 Hz, H-12), 3.366 (dd, 1H, J=9.5, 7.5 Hz, H-2′ of 20-Glc), 3.128 (dd, 1H, J=11.5, 4.5 Hz, H-3), 2.282 (td, 1H, J=10.5, 8.5 Hz, H-17), 2.14 (m, 2H, 6.8 Hz, H-23a), 2.018 (dq, 2H, J=20.5, 6.5 Hz, H-23b), 2.018 (dq, 2H, J=19.0, 6.5 Hz, H-22a), 1.734 (t, 1H, J=11.0 Hz, H-13), 1.673 (s, 3H, H-26), 1.616 (s, 3H, H-27), 1.359 (s, 3H, H-21), 1.216 (dt, 2H, J=13.0, 11.0 Hz, H-11β), 1.179 (dd, 2H, J=13.0, 5.5 Hz, H-11 α), 1.002 (s, 3H, H-29), 0.954 (s, 3H, H-28), 0.909 (s, 3H, H-30), O.905 (s, 3H, H-18), 0.769 (s, 3H, H-19).

CK-6′-monoglutarate (GS-17): ¹H-NMR (500 MHz, CD₃OD) δ: 5.11 (t, 1H, J=6.5 Hz, H-24), 4.58 (d, 1H, J=8, H-1′ of 20-Glc), 4.42 (dd, 2H, J=11.5, 1 Hz, H-6′ a of 20-Glc), 4.11 (dd, 2H, J=12, 7 Hz, H-6′b of 20-Glc), 3.71 (td, 1H, J=15.5, 5 Hz, H-12), 3.43 (broad, 1H, H-5′ of 20-Glc), 3.35 (t, 1H, J=9.5 Hz, H-3′ of 20-Glc), 3.23 (t,1H, J=9 Hz, H-4′ of 20-Glc), 3.14 (dd, 1H, J=11.5, 5 Hz, H-3), 3.11 (t, 1H, J=8.5 Hz, H-2′ of 20-Glc), 1.67 (s, 3H, H-26), 1.61 (s, 3H, H-27), 1.34 (s, 3H, H-21), 1.00 (s, 3H, H-29), 0.96 (s, 3H, H-28), 0.91 (s, 3H, H-30), 0.91 (s, 3H, H-18), 0.77 (s, 3H, H-19).

CK-3′-monoglutarate (GS-13): ¹H-NMR (500 MHz, CD₃OD) δ: 5.11 (t, 1H, J=7 Hz, H-24), 4.96 (t, 1H, J=9 Hz, H-3′ of 20-Glc), 4.70 (d, 1H, J=7.5, H-1′ of 20-Glc), 3.77 (dd, 2H, J=12, 2 Hz, H-6′a of 20-Glc), 3.66 (td, 1H, J=15.5, 5 Hz, H-12), 3.64 (dd, 2H, J=12, 5 Hz, H-6′b of 20-Glc), 3.47 (t,1H, J=9.5 Hz, H-4′ of 20-Glc), 3.29 (broad, 1H, H-5′ of 20-Glc), 3.20 (dd, 1H, J=9, 8 Hz, H-2′ of 20-Glc), 3.14 (dd, 1H, J=11.5, 5 Hz, H-3), 1.68 (s, 3H, H-26), 1.62 (s, 3H, H-27), 1.35 (s, 3H, H-21), 1.01 (s, 3H, H-29), 0.96 (s, 3H, H-28), 0.92 (s, 3H, H-30), 0.91 (s, 3H, H-18), 0.77 (s, 3H, H-19).

CK-3′,6′-diglutarate (GS-14): ¹H-NMR (500 MHz, CD₃OD) δ: 5.12 (t, 1H, J=7 Hz, H-24), 4.97 (t, 1H, J=9 Hz, H-3′ of 20-Glc), 4.69 (d, 1H, J=7.5, H-1′ of 20-Glc), 4.43 (dd, 2H, J=12, 2 Hz, H-6′a of 20-Glc), 3.71 (td, 1H, J=15.5, 5 Hz, H-12), 4.14 (dd, 2H, J=11.5, 6.5 Hz, H-6′b of 20-Glc), 3.55 (broad, 1H, H-5′ of 20-Glc), 3.41 (t, 1H, J=10 Hz, H-4′ of 20-Glc), 3.25 (dd, 1H, J=9.5, 8 Hz, H-2′ of 20-Glc), 3.14 (dd, 1H, J=11.5, 5 Hz, H-3), 1.68 (s, 3H, H-26), 1.62 (s, 3H, H-27), 1.36 (s, 3H, H-21), 1.01 (s, 3H, H-29), 0.96 (s, 3H, H-28), 0.92 (s, 3H, H-30), 0.91 (s, 3H, H-1 8), 0.78 (s, 3H, H-i 9).

CK-3′,4′,6′-triglutarate (GS-15): ¹H-NMR (500 MHz, CD₃OD) δ: 5.35 (t, 1H, J=9.5 Hz, H-3′ of 20-Glc), 5.12 (t, 1H, J=7 Hz, H-24), 4.90 (t, 1H, J=10 Hz, H-4′ of 20-Glc), 4.77 (d, 1H, J=8, H-1′ of 20-Glc), 4.17 (dd, 2H, J=12, 5.5 Hz, H-6′b of 20-Glc), 4.12 (dd, 2H, J=12, 2.5 Hz, H-6′a of 20-Glc), 3.81 (ddd, 1H, J=10, 5.5, 2.5,H-5′ of 20-Glc), 3.72 (td, 1H, J=15.5, 5 Hz, H-12), 3.22 (dd, 1H, J=9.5, 8 Hz, H-2′ of 20-Glc), 3.13 (dd, 1H, J=11.5, 5 Hz, H-3), 1.67 (s, 3H, H-26), 1.61 (s, 3H, H-27), 1.36 (s, 3H, H-21), 1.00 (s, 3H, H-29), 0.95 (s, 3H, H-28), 0.91 (s, 3H, H-30), 0.90 (s, 3H, H-18), 0.77 (s, 3H, H-19).

CK-3,2′,3′,4′,6′-pentaglutarate (GS-16): ¹H-NMR (500 MHz, CD₃OD) δ: 5.35 (t, 1H, J=9.5 Hz, H-3′ of 20-Glc), 5.12 (t, 1H, J=8 Hz, H-2′ of 20-Glc), 5.10 (t, 1H, J=7 Hz, H-24), 5.02 (t, 1H, J=10 Hz, H-4′ of 20- Glc), 4.90 (d, 1H, J=8, H-1′ of 20-Glc), 4.49 (dd, 1H, J=11, 5.5 Hz, H-3), 3.92 (broad, 1H, H-5′ of 20-Glc), 3.55 (td, 1H, J=15, 5 Hz, H-12), 1.67 (s, 3H, H-26), 1.62 (s, 3H, H-27), 1.34 (s, 3H, H-21), 1.00 (s, 3H, H-29), 0.94 (s,3H, H-28), 0.91 (s, 3H, H-30), 0.89 (s, 3H, H-18), 0.86 (s, 3H, H-19).

The following Table 1 and Table 2 separately list the ¹³C-NMR analysis results of the HPLC-purified CK and CK succinate and glutarate derivatives obtained from Examples 1 to 4.

TABLE 1
¹³C-NMR chemical shifts of CK and CK succinate derivatives

| | CK succinate derivatives | | | | |
|---|---|---|---|---|---|
| | | | | GS-9 | GS-11 |
| C atom | CK | GS-6 (mono-) | GS-8 (mono-) | (di-) | (tri-) |
| 1 | 40.24 | 40.25 | 40.26 | 40.26 | 40.25 |
| 2 | 27.99 | 28.01 | 28.00 | 28.00 | 28.00 |
| 3 | 79.52 | 79.57 | 79.49 | 79.57 | 76.83 |
| 4 | 40.03 | 40.03 | 40.03 | 40.02 | 40.02 |
| 5 | 57.25 | 57.29 | 57.25 | 57.28 | 57.28 |
| 6 | 19.41 | 19.42 | 19.43 | 19.41 | 19.41 |
| 7 | 35.84 | 35.85 | 35.85 | 35.85 | 35.85 |
| 8 | 40.96 | 40.95 | 40.96 | 40.95 | 40.96 |
| 9 | 51.05 | 51.11 | 51.06 | 51.11 | 51.11 |
| 10 | 38.17 | 38.18 | 38.17 | 38.17 | 38.17 |
| 11 | 30.99 | 30.83 | 30.94 | 30.75 | 30.75 |
| 12 | 71.93 | 71.71 | 71.82 | 71.65 | 71.65 |
| 13 | 49.76 | 49.69 | 49.76 | 49.71 | 49.74 |
| 14 | 52.49 | 52.45 | 52.45 | 52.41 | 52.41 |
| 15 | 31.64 | 31.52 | 31.61 | 31.47 | 31.46 |
| 16 | 27.19 | 27.29 | 27.20 | 27.26 | 27.28 |
| 17 | 53.14 | 53.00 | 52.98 | 52.86 | 52.75 |
| 18 | 16.23 | 16.29 | 16.29 | 16.31 | 16.31 |
| 19 | 16.72 | 16.67 | 16.74 | 16.66 | 16.64 |
| 20 | 84.94 | 85.00 | 85.20 | 85.35 | 85.63 |
| 21 | 22.85 | 22.29 | 22.74 | 22.16 | 22.20 |
| 22 | 36.65 | 36.77 | 36.65 | 36.66 | 36.64 |
| 23 | 24.26 | 23.82 | 24.11 | 23.67 | 23.66 |
| 24 | 125.84 | 126.04 | 125.78 | 125.99 | 125.90 |
| 25 | 132.30 | 132.21 | 132.25 | 132.26 | 132.33 |
| 26 | 25.89 | 25.91 | 25.91 | 25.91 | 25.90 |
| 27 | 17.95 | 17.89 | 17.97 | 17.89 | 17.88 |
| 28 | 28.61 | 28.63 | 28.64 | 28.63 | 28.63 |
| 29 | 16.13 | 16.12 | 16.16 | 16.12 | 16.12 |
| 30 | 17.17 | 17.32 | 17.25 | 17.35 | 17.36 |
| | | | | | 3′ & 4′ |
| 20-O-Glc | | 6′ | 3′ | 3′ & 6′ | & 6′ |
| 1′ | 98.30 | 97.99 | 98.01 | 97.74 | 97.72 |
| 2′ | 75.40 | 75.17 | 73.54 | 73.46 | 72.69 |
| 3′ | 78.19 | 78.46 | 79.66 | 79.76 | 79.57 |
| 4′ | 71.17 | 71.71 | 69.21 | 69.86 | 70.10 |
| 5′ | 77.95 | 75.31 | 77.56 | 74.79 | 73.39 |
| 6′ | 62.50 | 65.11 | 62.20 | 64.78 | 63.89 |
| | | | | GS-9 | GS-11 |
| succinate | CK | GS-6 (mono-) | GS-8 (mono-) | (di-) | (tri-) |
| 3′-S1 | | | 173.90 | 173.91 | 173.24 |
| 3′-S2 | | | 29.94 | 29.79 | 29.55 |
| 3′-S3 | | | 30.29 | 30.05 | 29.98 |
| 3′-S4 | | | 176.28 | 175.87 | 175.84 |
| 4′-S1 | | | | | 173.71 |
| 4′-S2 | | | | | 29.60 |
| 4′-S3 | | | | | 30.02 |
| 4′-S4 | | | | | 175.89 |
| 6′-S1 | | 174.05 | | 173.98 | 173.73 |
| 6′-S2 | | 29.97 | | 29.89 | 29.79 |
| 6′-S3 | | 30.16 | | 30.24 | 30.05 |
| 6′-S4 | | 176.06 | | 176.41 | 176.10 |

TABLE 2
¹³C-NMR chemical shifts of CK glutarate derivatives

| | CK glutarate derivatives | | | | |
|---|---|---|---|---|---|
| | | | GS-14 | GS-15 | GS-16 |
| C atom | GS-17 (mono-) | GS-13 (mono-) | (di-) | (tri-) | (penta-) |
| 1 | 40.24 | 40.27 | 40.24 | 40.25 | 39.63 |
| 2 | 28.00 | 28.00 | 28.02 | 28.02 | 27.33 |
| 3 | 79.56 | 79.53 | 79.22 | 79.53 | 82.28 |
| 4 | 40.02 | 40.03 | 40.03 | 40.03 | 38.99 |
| 5 | 57.27 | 57.26 | 57.26 | 57.26 | 57.11 |
| 6 | 19.41 | 19.41 | 19.43 | 19.43 | 19.26 |
| 7 | 35.84 | 35.85 | 35.85 | 35.85 | 35.66 |
| 8 | 40.94 | 40.96 | 40.95 | 40.95 | 40.94 |
| 9 | 51.09 | 51.06 | 51.08 | 51.10 | 50.77 |
| 10 | 38.17 | 38.17 | 38.17 | 38.17 | 38.14 |
| 11 | 30.81 | 30.96 | 30.78 | 30.77 | 30.83 |
| 12 | 71.68 | 71.88 | 71.62 | 71.62 | 71.90 |
| 13 | 49.68 | 49.77 | 49.69 | 49.72 | 49.63 |
| 14 | 52.43 | 52.47 | 52.40 | 52.40 | 52.48 |
| 15 | 31.50 | 31.61 | 31.48 | 31.45 | 31.41 |
| 16 | 27.28 | 27.19 | 27.27 | 27.29 | 25.93 |
| 17 | 52.97 | 53.06 | 52.90 | 52.78 | 53.82 |
| 18 | 16.29 | 16.27 | 16.32 | 16.33 | 16.66 |
| 19 | 16.66 | 16.72 | 16.67 | 16.67 | 17.07 |
| 20 | 84.99 | 85.25 | 85.31 | 85.61 | 86.45 |
| 21 | 22.23 | 22.79 | 22.18 | 22.21 | 22.51 |
| 22 | 36.72 | 36.63 | 36.66 | 36.64 | 36.80 |
| 23 | 23.74 | 24.16 | 23.68 | 23.66 | 23.96 |
| 24 | 126.07 | 125.82 | 125.94 | 125.85 | 125.67 |
| 25 | 132.17 | 132.33 | 132.15 | 132.23 | 132.44 |
| 26 | 25.94 | 25.90 | 25.94 | 25.94 | 24.72 |
| 27 | 17.91 | 17.96 | 17.93 | 17.93 | 17.98 |
| 28 | 28.63 | 28.63 | 28.64 | 28.64 | 28.55 |
| 29 | 16.13 | 16.13 | 16.14 | 16.14 | 16.24 |
| 30 | 17.33 | 17.22 | 17.36 | 17.38 | 17.30 |
| | | | | 3′, | 3, 2′, 3′, |
| 20-O-Glc | 6′ | 3′ | 3′ & 6′ | 4′, 6′ | 4′, 6′ |
| 1′ | 97.95 | 98.15 | 97.78 | 97.72 | 95.65 |
| 2′ | 75.28 | 73.64 | 73.50 | 72.65 | 72.85 |
| 3′ | 78.50 | 79.08 | 79.53 | 76.79 | 74.25 |
| 4′ | 71.75 | 69.25 | 69.91 | 70.09 | 69.83 |
| 5′ | 75.08 | 77.71 | 74.85 | 73.35 | 73.05 |
| 6′ | 64.96 | 62.21 | 64.60 | 63.53 | 63.37 |
| | | | GS-14 | GS-15 | GS-16 |
| glutarate | GS-17 (mono-) | GS-13 (mono-) | (di-) | (tri-) | (penta-) |
| 3-G1 | | | | | 172.90 |
| 3-G2 | | | | | 33.76 |
| 3-G3 | | | | | 21.11 |
| 3-G4 | | | | | 33.87 |
| 3-G5 | | | | | 176.31 |
| 2′-G1 | | | | | 173.21 |
| 2′-G2 | | | | | 33.76 |
| 2′-G3 | | | | | 21.11 |
| 2′-G4 | | | | | 33.93 |
| 2′-G5 | | | | | 176.45 |
| 3′-G1 | | 174.56 | 174.35 | 173.38 | 173.61 |
| 3′-G2 | | 33.88 | 34.04 | 33.84 | 33.81 |
| 3′-G3 | | 21.36 | 21.36 | 21.13 | 21.16 |
| 3′-G4 | | 34.31 | 34.04 | 33.96 | 33.93 |
| 3′-G5 | | 176.98 | 176.69 | 176.60 | 176.51 |
| 4′-G1 | | | | 174.05 | 174.13 |
| 4′-G2 | | | | 33.92 | 33.83 |
| 4′-G3 | | | | 21.23 | 21.18 |
| 4′-G4 | | | | 33.96 | 33.99 |
| 4′-G5 | | | | 176.67 | 176.63 |
| 6′-G1 | 174.53 | | 174.45 | 174.12 | 174.58 |
| 6′-G2 | 33.97 | | 34.04 | 33.92 | 33.87 |
| 6′-G3 | 21.30 | | 21.39 | 21.23 | 21.52 |
| 6′-G4 | 34.05 | | 34.28 | 34.05 | 34.57 |
| 6′-G5 | 176.70 | | 177.09 | 176.77 | 176.63 |

Experiment 1 (Control): CK Anti-Cancer Activity Assays

Four human tumor cell lines, OVCAR-3 (Adenocarcinoma, ovary, human), A549 (Carcinoma, lung, human), HT-29 (Adenocarcinoma, colon, moderately well-differentiated grade II, human) and MCF-7 (Breast adenocarcinoma, pleural effusion, human) were chosen in the experiments., which were obtained from American Type Culture Collection (ATCC) under codes of ATCC HTB-161, ATCC CCL-185, ATC HTB-38 and ATCC HTB-22, respectively. The culture medium used for OVCAR-3 was RPMI 1640 medium with 20% fetal bovine serum, which was supplemented with 0.01 mg bovine insulin per ml and 1% Antibiotice-Antimycotic. The culture medium used for A549 was F-12K nutrient mixture (Kaighn's Modification), 90%; and fetal bovine serum 10%. The culture medium used for HT-29 was McCoy's 5A medium, 90%; and fetal bovine serum, 10%. The culture medium used for MCF-7 was Minimum Essential Medium, 90%; and fetal bovine serum, 10%.

CK was dissolved in a mixed solvent of ethanol and Cremophor® RH40 (ethanol:Cremophor® RH40=1:1), and then diluted with sterile distilled water to obtain test solutions having CK concentrations of 10000, 1000, 100, 10, 1 μg/ml in 5% ethanol and 5% Cremophor® RH40.

Aliquots of 100 μl of cell suspension (about 1.0˜3.0×10³/well) were placed in 96-well microtiter plates in an atmosphere of 5% CO₂ at 37° C. After 24 hours, 100 μl of growth medium and 2 μl of test solution or vehicle (5% ethanol and 5% Cremophor® RH40) were added per respectively per well in duplicate for an additional 72-hour incubation. Thus, the final concentration of vehicle was 0.05%. The test compound, CK, was evaluated at concentrations of 100, 10, 1, 0.1, 0.01 μg/ml. At the end of the incubation, 20 μl of alamarBlue 90% reagent was added to each well for another 6-hour incubation before detection of cell viability by fluorescent intensity. Fluorescent intensity was measured using a SPECTRAfluor Plus plate reader with excitation at 530 nm and emission at 590 nm.

Experiment 2: CK Succinate and CK Glutarate Anti-Cancer Activity Assays

The tumor cell lines used were the same as in Experiment 1.

CK succinate and CK glutarate derivatives were directly dissolved in the phosphate buffered saline (PBS) having a pH of 7.4, and then diluted with sterile distilled water to obtain test solutions having concentrations of the CK derivative of 2000, 200, 20, 2, 0.2 μg/ml in 40% PBS

Aliquots of 100 μl of cell suspension (about 5.0×10³/well) were placed in 96-well microtiter plates in an atmosphere of 5% CO₂ at 37° C. After 24 hours, 90 μl of growth medium and 10 μl of test solution or vehicle (40% PBS, pH 7.4) were added per respectively per well in duplicate for an additional 72-hour incubation. Thus, the final concentration of vehicle was 0.05%. The test compound, CK derivative, was evaluated at concentrations of 100, 10, 1, 0.1, 0.01 μg/ml. At the end of the incubation, 20 μl of alamarBlue 90% reagent was added to each well for another 6-hour incubation before detection of cell viability by fluorescent intensity. Fluorescent intensity was measured using a SPECTRAfluor Plus plate reader with excitation at 530 nm and emission at 590 nm.

The measured results were calculated according to the following formulas:
PG (Percent Growth):
   PG(%)=100%×(Mean Fₜₑₛₜ-Mean Fₜᵢₘₑ₀)/(Mean F_{ctrl}-Mean Fₜᵢₘₑ₀)
If (Mean Fₜₑₛₜ-Mean Fₜᵢₘₑ₀)<0, then
   PG(%)=100%×(Mean Fₜₑₛₜ-Mean Fₜᵢₘₑ₀)/(Mean Fₜᵢₘₑ₀-Mean F_{blank})
   wherein
Mean Fₜᵢₘₑ₀=The average of two measured fluorescent intensities of reduced alamarBlue at the time just before exposure of cells to the test compound;
Mean Fₜₑₛₜ=The average of two measured fluorescent intensities of alamarBlue after 72-hour exposure of cells to the test compound;
Mean F_{ctrl}=The average of two measured fluorescent intensities of alamarBlue after 72-hour incubation without the test compound;
Mean F_{blank}=The average of two measured fluorescent intensities of alamarBlue in medium without cells after 72-hour incubation.

50% Inhibition concentration (IC₅₀): Test compound concentration where the increase in the number or mass of treated cells from time₀ was only 50% as much as the corresponding increase in the vehicle control at the end of experiment. IC₅₀ was determined by nonlinear regression using GraphPd Prism (GraphPad Software, USA).

IC₅₀ of the test compounds including CK and some of the CK succinate, and glutarate derivatives purified by HPLC in Examples 1 to 3 are listed in Table 3.

TABLE 3
IC₅₀ (μM) of CK, CK succinate and CK glutarate derivatives

| | GS-12 | | | | | | |
|---|---|---|---|---|---|---|---|
| | (CK) | GS-6 | GS-8 | GS-9 | GS-11 | GS-13 | GS-14 |
| OVCAR-3 | 2.7 μM | 22.1 μM | 19.3 μM | 89.9 μM | >100 μM | 69.1 μM | 79.8 μM |
| MCF-7 | 11.2 μM | | 53.9 μM | | | | |
| HT-29 | 9.3 μM | | 71.9 μM | | | | |
| A549 | 17.7 μM | | 31.8 μM | | | | |

The CK succinate and glutarate derivatives purified by HPLC in Examples 1 to 3 were dissolved in PBS to observe their solubility, and the results show that all the CK succinate and glutarate derivatives have a solubility greater than 10 mg/ml in PBS.

### Example 5

### Synthesis of PPD Succinate

100 mg of PPD was dissolved in 8 ml of dried THF, and 217.1 mg (10 equivalents) of succinic anhydride was added to the resulting solution while stirring, to which 265.2 mg (10 equivalents) of 4-(dimethylamino)pyridine was then added. The reaction mixture was then refluxed under N₂ for 24 hours. The reaction progress was monitored by reversed-phase TLC, and the solvent was removed by rotary evaporation until dryness, when the reaction was completed. 10 ml of 0.01 M HCl was then added to the residue followed by the addition of 15 ml of ethyl acetate. The mixture was then transferred to a seperatory fumnel. Drained the aqueous layer, and washed the organic layer once with 10 ml of 0.01 M HCl. The organic layer was then wash with Brine, dried over sodium sulfate and rotary evaporated to dryness.

The obtained product was analyzed by HPLC where the mobile phase was 72% acetonitrile/H₂O (containing 0.02% phosphoric acid). The elution times of the main products separately were: 38.1 min, 40.3 min, 46.1 min, and 48.4 min. A semi-preparative HPLC was used for purification separation, thereby obtaining 5.6 mg of 38.1 min product (GS41) 39.9 mg of 40.3 min product (GS42), 1.9 mg of 46.1 min product (GS43), and 6.3 mg of 48.4 min product (GS44). The four products GS41 to GS44 were identified by mass spectrum (MS) as a PPD monosuccinate derivative, PPD disuccinate derivateive, PPD and PPD monosuccinate derivative, respectively. Verifications from ¹³C-NMR and ¹H-NMR indicated that GS41 has a monosuccinate which substitutes the hydrogen of the OH group at 12^{th} of PPD; and GS42 has two succinates which substitute the hydrogens of the OH groups at 3^{rd} and 12^{th} of PPD.

¹³C-NMR chemical shifts of PPD and PPD-succinate derivatives

| | PPD-succinate derivatives | | |
|---|---|---|---|
| C atom | PPD | GS41 (mono-) | GS42 (di-) |
| 1 | 40.26 | 40.01 | 39.49 |
| 2 | 28.00 | 27.94 | 27.60 |
| 3 | 79.51 | 79.44 | 82.37 |
| 4 | 40.03 | 40.04 | 40.88 |
| 5 | 57.27 | 57.21 | 57.15 |
| 6 | 19.41 | 19.39 | 19.20 |
| 7 | 35.92 | 35.66 | 35.52 |
| 8 | 40.94 | 40.87 | 40.88 |
| 9 | 51.37 | 51.32 | 51.13 |
| 10 | 38.20 | 38.22 | 38.17 |
| 11 | 31.99 | 29.49 | 29.48 |
| 12 | 72.11 | 76.87 | 76.78 |
| 13 | 48.89 | 46.64 | 46.61 |
| 14 | 52.57 | 53.23 | 53.22 |
| 15 | 31.99 | 32.28 | 32.27 |
| 16 | 27.36 | 27.61 | 27.60 |
| 17 | 55.13 | 53.67 | 53.64 |
| 18 | 16.16 | 16.08 | 16.99 |
| 19 | 16.75 | 16.66 | 16.64 |
| 20 | 74.38 | 75.25 | 75.24 |
| 21 | 26.51 | 25.91 | 24.53 |
| 22 | 36.30 | 38.81 | 38.75 |
| 23 | 23.28 | 23.45 | 23.44 |
| 24 | 126.18 | 126.19 | 126.18 |
| 25 | 131.97 | 132.03 | 132.02 |
| 26 | 25.93 | 25.94 | 25.93 |
| 27 | 17.71 | 17.78 | 17.78 |
| 28 | 28.62 | 28.59 | 28.68 |
| 29 | 16.12 | 16.08 | 16.08 |
| 30 | 17.11 | 17.99 | 17.99 |
| succinate | | 12 | 3&12 |
| 12-S1 | | 173.61 | 173.57 |
| 12-S2 | | 29.77 | 29.77 |
| 12-S3 | | 30.77 | 30.52 |
| 12-S4 | | 176.01 | 175.99 |
| 3-S1 | | | 173.97 |
| 3-S2 | | | 29.73 |
| 3-S3 | | | 30.77 |
| 3-S4 | | | 176.2 |

PPD: ¹H-NMR (500 MHz, CD₃OD) δ: 5.134 (t, 1H, H-24), 3.536 (td, 1H, H012), 3.137 (dd, 1H, H-3).

PPD-12-monosuccinate (GS41): ¹H-NMR (500 MHz, CD₃OD) δ: 5.137 (t, 1H, H-24), 4.862 (td, 1H, H-12), 3.140 (dd, 1H, H-3).

PPD-3,12-disuccinate (GS42): ¹H-NMR (500 MHz, CD₃OD) δ: 5.13 (t, 1H, H-24), 4.87 (dt, 1H, H-12), 4.48 (dd, 1H, H-3).

### Example 6

### Synthesis of PPT Succinate

50 mg of PPT was dissolved in 4 ml of dried THF, and 104.9 mg (10 equivalents) of succinic anhydride was added to the resulting solution while stirring, to which 4 ml of Et₃N was then added. The resulting reaction mixture was then refluxed under N₂ for 48 hours. This reaction progress was monitored by reversed-phase TLC, and the solvent was removed by rotary evaporation until dryness, when the reaction was completed. 10 ml of 0.01M HCl was then added to the residue followed by the addition of 15 ml of ethyl acetate. The mixture was then transferred to a seperatory funnel. Drained the aqueous layer, and washed the organic layer once with 10 ml of 0.01M HCR. The organic layer was then wash with Brine, dried over sodium sulfate and rotary evaporated to dryness.

The obtained product was analyzed by HPLC where the mobile phase was 50% acetonitrile/H₂O (containing 0.02% phosphoric acid). The elution times of the main products separately were: 18.3 min, 19.3 min, 26.3 min, and 32.3 min. A semi-preparative HPLC was used for purification separation, thereby obtaining 1.5 mg of 18.3 min product (GS54), 3.5 mg of 19.3 min product (GS51), 28.8 mg of 26.3 min product (GS53), and 6.6 mg of 32.3 min product (GS52). The four products GS51 to GS54 were identified by mass spectrum (MS) as a PPT monosuccinate derivateive, PPT disuccinate derivative, PPT trisuccinate derivateive and PPT disuccinate derivative, respectively. Verifications from ¹³C-NMR and ¹H-NMR indicated that GS51 has monosuccinate which substitutes the hydrogen of the OH group at 12^{th} of PPT; GS52 has two succinates which substitute the hydrogens of the OH group at 3^{rd} and 12^{th} of PPT; GS53 has three succinates which substitute the hydrogens of the OH group at 3^{rd}, 6^{th} and 12^{th} of PPT; and GS54 has two succinates which substitute the hydrogens of the OH group at 6^{th} and 12^{th} of PPT.

¹³C-NMR chemical shifts of PPT and PPT-succinate derivatives

| | PPT-succinate derivatives | | | | |
|---|---|---|---|---|---|
| | | | GS52 | | |
| C atom | PPT | GS51 (mono-) | (di-) | GS53 (tri-) | GS54 (di-) |
| 1 | 40.03 | 39.83 | 39.26 | 38.95 | 39.95 |
| 2 | 27.70 | 27.66 | 29.43 | 29.33 | 27.53 |
| 3 | 79.45 | 79.41 | 82.53 | 81.89 | 78.82 |
| 4 | 40.45 | 41.81 | 41.82 | 41.82 | 41.80 |
| 5 | 62.07 | 62.07 | 62.02 | 59.75 | 59.89 |
| 6 | 68.86 | 68.83 | 68.57 | 71.98 | 72.28 |
| 7 | 47.19 | 46.95 | 46.89 | 46.24 | 46.20 |
| 8 | 41.94 | 46.20 | 46.23 | 43.11 | 43.16 |
| 9 | 50.69 | 50.62 | 50.10 | 50.41 | 50.55 |
| 10 | 40.10 | 40.45 | 40.06 | 40.46 | 40.49 |
| 11 | 31.82 | 31.33 | 32.18 | 32.12 | 31.16 |
| 12 | 71.96 | 76.52 | 76.49 | 76.28 | 76.36 |
| 13 | 49.57 | 49.57 | 49.28 | 49.29 | 49.57 |
| 14 | 52.38 | 53.20 | 53.29 | 53.23 | 53.18 |
| 15 | 31.36 | 31.13 | 31.21 | 31.04 | 31.12 |
| 16 | 26.40 | 25.89 | 25.95 | 25.96 | 25.90 |
| 17 | 55.08 | 53.42 | 53.44 | 53.41 | 53.38 |
| 18 | 17.52 | 17.71 | 17.77 | 17.77 | 17.71 |
| 19 | 17.04 | 17.54 | 17.61 | 17.59 | 17.52 |
| 20 | 74.30 | 75.13 | 75.16 | 75.14 | 75.10 |
| 21 | 27.27 | 27.50 | 27.78 | 27.50 | 27.46 |
| 22 | 36.19 | 38.75 | 38.74 | 38.73 | 38.71 |
| 23 | 23.21 | 23.40 | 23.44 | 23.44 | 23.40 |
| 24 | 126.10 | 126.13 | 126.18 | 126.18 | 126.10 |
| 25 | 131.94 | 131.98 | 132.04 | 132.04 | 131.99 |
| 26 | 25.84 | 25.86 | 25.92 | 25.92 | 25.85 |
| 27 | 17.64 | 17.89 | 17.95 | 17.89 | 17.82 |
| 28 | 31.91 | 32.15 | 39.47 | 38.98 | 32.08 |
| 29 | 15.71 | 16.03 | 17.02 | 17.21 | 16.23 |
| 30 | 16.05 | 17.46 | 17.50 | 17.29 | 17.24 |
| | | | | 3 & 6 | |
| succinate | | 12 | 3 & 12 | & 12 | 6 & 12 |
| 12-S1 | | 173.80 | 173.67 | 173.55 | 173.62 |
| 12-S2 | | 29.37 | 30.03 | 29.72 | 29.98 |
| 12-S3 | | 30.70 | 30.86 | 30.55 | 30.69 |
| 12-S4 | | 176.89 | 176.16 | 175.90 | 176.10 |
| 6-S1 | | | | 173.63 | 173.66 |
| 6-S2 | | | | 29.82 | 29.92 |
| 6-S3 | | | | 30.80 | 30.86 |
| 6-S4 | | | | 175.98 | 176.25 |
| 3-S1 | | | 174.14 | 173.99 | |
| 3-S2 | | | 29.94 | 29.88 | |
| 3-S3 | | | 30.76 | 30.98 | |
| 3-S4 | | | 176.20 | 176.06 | |

PPT: ¹H-NMR (500 MHz, CD₃OD) δ: 5.13 (t, 1H, H-24), 4.02 (dt, 1H, H-6), 3.56 (dt, 1H, H-12), 3.11 (dd, 1H, H-3), 2.03 (q, 2H, H-23ab), 1.88 (broad, 1H, H-16a), 1.84 (2H, H-11a), 1.69 (s, 3H, H-26), 1.61 (s, 3H, H-27), 1.61 (s, 3H, H-18), 1.28 (s, 3H, H-28), 1.15 (s, 3H, H-21), 1.07 (s, 3H, H-i 9), 0.95 (s, 3H, H-30), 0.94 (s, 3H, H-29).

PPT-12-monosuccinate (GS51): ¹H-NMR (500 MHz, CD₃OD) δ: 5.13 (t, 1H, H-24), 4.8 (1H, H-12), 4.01 (dt, 1H, H-6), 3.11 (dd, 1H, H-3), 1.85 (2H, H-11a), 1.68 (s, 3H, H-26), 1.62 (s, 3H, H-27), 1.62 (s, 3H, H-18), 1.28 (s, 3H, H-28), 1.13 (s, 3H, H-21), 1.09 (s, 3H, H-19), 1.00 (s, 3H, H-30), 0.94 (s, 3H, H-29).

PPT-3,12-disuccinate (GS52): ¹H-NMR (500 MHz, CD₃OD) δ: 5.13 (t, 1H, H-24), 4.8 (1H, H-12), 4.57 (dd, 1H, H-3), 4.03 (dt, 1H, H-6), 1.68 (s, 3H, H-26), 1.62 (s, 3H, H-27), 1.28 (s, 3H, H-18), 1.13 (s, 3H, H-28), 1.07(s, 3H, H-19), 1.01 (s, 3H, H-30), 0.96 (s, 3H, H-29).

PPT-3,6,12-trisuccinate (GS53): ¹H-NMR (500 MHz, CD₃OD) δ: 5.39 (dt, 1H, H-6), 5.13 (t, 1H, H-24), 4.8 (1H, H-12), 4.11 (dd, 1H, H-3), 1.68 (s, 3H, H-26), 1.62 (s, 3H, H-27), 1.14 (s, 3H, H-18), 1.12 (s, 3H, H-28), 1.07 (s, 3H, H-21), 1.02 (s, 3H, H-19), 1.00 (s, 3H, H-30), 0.93 (s, 3H, H-29).

PPT-6,12-disuccinate (GS54): ¹H-NMR (500 MHz, CD₃OD) δ: 5.38 (dt, 1H, H-6), 5.13 (t, 1H, H-24), 4.8 (1H, H-12), 3.14 (dd, 1H, H-3), 1.84 (2H, H-11a), 1.68 (s, 3H, H-26) 1.62 (s, 3H, H-27), 1.28 (s, 3H, H-18), 1.17 (s, 3H, H-28), 1.13 (s, 3H, H-21), 1.12 (s, 3H, H-19), 0.99 (s, 3H, H-30), 0.98 (s, 3H, H-29).

### Example 7

### Synthesis of F1 Succinate

400 mg of F1 and 249.6 mg (4 equivalents) of succinic anhydride were dissolved in a mixed solution of 48 ml Et₃N and 16 ml THF, and the resulting solution was kept at room temperature (27° C.) under oscillation for 2˜15 hours to react. White precipitate was formed and recovered by filtration, which was then dried by removing the solvent therefrom in vacuo. The obtained product was analyzed by HPLC where the mobile phase was 40% acetonitrile/H₂O (containing 0.02% phosphoric acid). The elution times of the main products separately were: 9.4 min, 13.6 min, 17.3 min, 25.3 min and 36.1 min. A semi-preparative HPLC was used for purification separation, thereby obtaining 65 mg of 9.4 min product (F1), 10 mg of 13.6 min product (GS31), 262 mg of 17.3 min product (GS32), 24 mg of 25.3 min product (GS33) and 14 mg of 36.1 min product (GS34). The products GS32 and GS33 were identified by mass spectrum (MS) as a F1 monosuccinate derivative and F1 disuccinate derivateive, respectively. Verifications from ¹³C-NMR and ¹H-NMR indicated that GS32 has monosuccinate which substitutes the hydrogen at 3^{rd} OH groups (3′) on the glucose group at 20^{th} of F1; and GS33 has two succinates which substitute the hydrogens at 3^{rd} and 6^{th} OH groups (3′,6′) on the glucose group at 20^{th} of F 1.

¹³C-NMR chemical shifts of F1 and F1-succinate derivatives

| | F1-succinate derivatives | | |
|---|---|---|---|
| C atom | F1 | GS32 (mono-) | GS33 (di-) |
| 1 | 40.10 | 40.12 | 40.10 |
| 2 | 30.91 | 30.79 | 30.66 |
| 3 | 78.27 | 79.50 | 79.57 |
| 4 | 47.20 | 47.18 | 47.20 |
| 5 | 62.13 | 62.13 | 62.15 |
| 6 | 68.89 | 68.86 | 68.92 |
| 7 | 40.14 | 40.16 | 40.14 |
| 8 | 42.02 | 41.99 | 42.01 |
| 9 | 50.46 | 50.47 | 50.51 |
| 10 | 40.50 | 40.49 | 40.50 |
| 11 | 31.44 | 31.44 | 31.45 |
| 12 | 71.80 | 71.67 | 71.54 |
| 13 | 49.41 | 49.37 | 49.29 |
| 14 | 52.36 | 52.30 | 52.28 |
| 15 | 31.62 | 31.54 | 31.45 |
| 16 | 27.19 | 27.16 | 27.24 |
| 17 | 53.11 | 52.86 | 52.80 |
| 18 | 17.67 | 17.68 | 17.62 |
| 19 | 17.67 | 17.70 | 17.75 |
| 20 | 84.89 | 85.18 | 85.31 |
| 21 | 22.80 | 22.63 | 22.13 |
| 22 | 36.62 | 36.57 | 36.60 |
| 23 | 24.21 | 24.02 | 23.64 |
| 24 | 125.85 | 125.83 | 125.98 |
| 25 | 132.30 | 132.31 | 132.27 |
| 26 | 25.86 | 25.90 | 25.91 |
| 27 | 17.93 | 17.95 | 17.89 |
| 28 | 27.76 | 27.73 | 27.76 |
| 29 | 16.12 | 16.13 | 16.12 |
| 30 | 17.21 | 17.30 | 17.39 |
| 20-O-Glc | F1 | GS32 (mono-) | GS33 (di-) |
| 1′ | 98.31 | 97.99 | 97.73 |
| 2′ | 75.40 | 73.50 | 73.45 |
| 3′ | 79.54 | 79.76 | 79.80 |
| 4′ | 71.23 | 69.24 | 69.87 |
| 5′ | 77.95 | 77.56 | 74.79 |
| 6′ | 62.55 | 62.24 | 64.78 |
| succinate | | 3′ | 3′&6′ |
| 3′-S1 | | 173.98 | 173.91 |
| 3′-S2 | | 29.89 | 29.80 |
| 3′-S3 | | 30.28 | 30.07 |
| 3′-S4 | | 176.31 | 175.88 |
| 6′-S1 | | | 173.99 |
| 6′-S2 | | | 29.92 |
| 6′-S3 | | | 30.28 |
| 6′-S4 | | | 176.41 |

F1: ¹H-NMR (500 MHz, CD₃OD) δ: 5.11 (t, 1H, H-24), 3.35 (t, 1H, H-3′ of 20-Glc), 4.60 (d, 1H, H-1′ of 20-Glc), 4.02 (dt, 1H, H-6), 3.78 (dd, 2H, H-6′b of 20-Glc), 3.69 (dd, 2H, H-6′a of 20-Glc), 3.64 (dt, 1H, H-12), 3.32 (t, 1H, H-4′ of 20-Glc), 3.21 (broad, 1H, H-5′ of 20-Glc), 3.07 (t, 1H, H-2′ of 20-Glc), 3.11 (dd, 1H, H-3), 2.28 (q, 1H, H-17), 2.08 (q, 2H, H-23ab), 1.92 (broad, 1H, H-16a), 1.84 (2H, H-11a), 1.68 (s, 3H, H-26), 1.62 (s, 3H, H-27), 1.34 (s, 3H, H-21), 1.39 (s, 3H, H-28), 0.96 (s, 3H, H-29), 0.96 (s, 3H, H-30).

F1-3′-monosuccinate (GS32): ¹H-NMR (500 MHz, CD₃OD) δ: 5.11 (t, 1H, H-24), 4.96 (t, 1 H, H-3′ of 20-Glc), 4.71 (d, 1H, H-1′ of 20-Glc), 4.02 (dt, 1H, H-6), 3.79 (dd, 2H, H-6′b of 20-Glc), 3.71 (d, 2H, H-6′a of 20-Glc), 3.67 (dt, 11H, H-1 2), 3.49 (t, 1H, H-4′ of 20-Glc), 3.30 (broad, 1H, H-5′ of 20-Glc), 3.22 (t, 1H, H-2′ of 20-Glc), 3.12 (dd, 1H, H-3), 2.28 (q, 1H, H-17), 2.09 (q, 2H, H-23ab), 1.92 (dt, 1H, H-16a), 1.84 (2H, H-11a), 1.82 (2H, H-22a), 1.68 (s, 3H, H-26), 1.63 (s, 3H, H-27), 1.36 (s, 3H, H-21), 1.29 (s, 3H, H-28), 0.96 (s, 3H, H-29), 0.96 (s, 3H, H-30).

F1-3′,6′-disuccinate (GS33): ¹H-NMR (500 MHz, CD₃OD) δ: 5.12 (t, 1H, H-24), 4.96 (t, 1H, H-3′ of 20-Glc), 4.69 (d, 1H, H-1′ of 20-Glc), 4.14 (dd, 2H, H-6′ab of 20-Glc), 4.02 (dt, 1H, H-6), 3.73 (dt, 11H, H-12), 3.54 (t,lH, H-4′ of 20-Glc), 3.42 (t, 1H, H-5′ of 20-Glc), 3.26 (t, 1H, H-2′ of 20-Glc), 3.11 (dd, 1H, H-3), 2.28 (q, 1H, H-17), 1.67 (s, 3H, H-26), 1.62 (s, 3H, H-27), 1.36 (s, 3H, H-21), 1.28 (s, 3H, H-28), 0.95 (s, 3H, H-29), 0.95 (s, 3H, H-30).

IC₅₀ on OVCAR-3 tumor cell of some of the ginsenosides and succinate derivatives synthesized in Examples 5 to 7 are listed in Table 4.

TABLE 4
IC₅₀ (μM) of ginsenosides and it's succinate derivatives on OVCAR-3
tumor cell

| | GS30 | | GS40 | | | GS50 | | | |
|---|---|---|---|---|---|---|---|---|---|
| | (F1) | GS32 | (PPD) | GS41 | GS42 | (PPT) | GS51 | GS52 | GS53 |
| IC₅₀ | 50.1 | 14.9 | 28.2 | 42.8 | 30.3 | 31.5 | 31.2 | 26.6 | 47.6 |

Although the present invention has been described with reference to specific details of certain embodiments thereof, it is not intended that such details should be regarded as limitations upon the scope of the invention. Many modifications and variations are possible in light of the above disclosure.

1. A dicarboxylic acid ester of ginsenoside.

2. The dicarboxylic acid ester of ginsenoside of claim 1 comprising at least one group of —OC(O)—A—C(O)OH which replaces —OH of the ginsenoside, wherein A is —(CR⁴R⁹)ₙ—, wherein R⁴ and R⁹ independently are H or C1-C4 alkyl, and n is an integer of 0-8.

3. The dicarboxylic acid ester of ginsenoside of claim 2, which is represented by the following formulas (I), (II), (III-A) or (III-B): 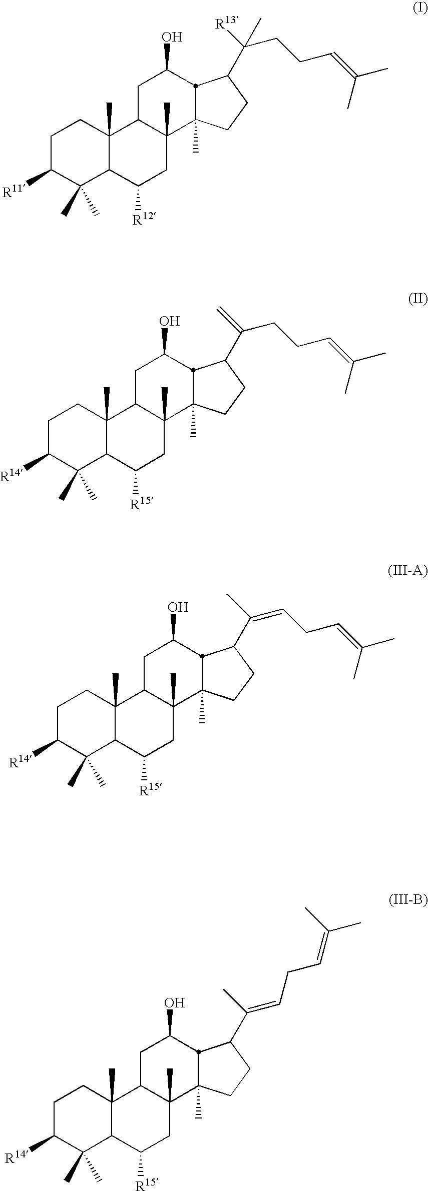
wherein R^{11′} is R¹¹, —OE, —O—Glc′ or —O—Glc—Glc′; R^{12′} is R¹², —OE, —O—Glc′, —O—Glc—Glc′ or —O—Glc—Rha′; R^{13′} is R¹³, —OE or —O—Glc′; R^{14′} is R¹⁴, —OE, —O—Glc′, —O—Glc—Glc′, or —O—Glc—Glc—Ac′; and R^{15′} is R¹⁵, —O—Glc′, —O—Glc—Ac′, or —O—Glc—Rha′, wherein
E is —C(O)(CR⁴R⁹)ₙCOOH, wherein R⁴, R⁹ and n are defined as in claim 2;
—O—Glc′ is 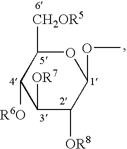
wherein R⁵, R⁶, R⁷ and R⁸ are H or E, wherein E is defined as above;
—O—Glc—Glc′ is 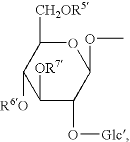
wherein R^{5′}, R^{6′}, and R^{7′} are H or E, wherein E and —O—Glc′ are defined as above;
—O—Glc—Rha′ is 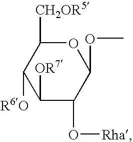
wherein R^{5′}, R^{6′}, and R^{7′} are H or E, wherein E is defined as above, and —O—Rha′ is 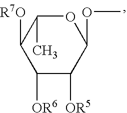
wherein R⁵, R⁶, and R⁷ are defined as above;
—O—Glc—Ac′ is 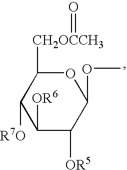
wherein R⁵,R⁶, and R⁷ are defined as above;
—O—Glc—Glc—Ac′ is 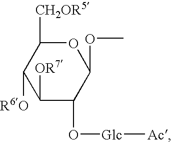
wherein R^{5′}, R^{6′}, R^{7′} and —O—Glc—Ac′ are defined as above;
wherein R¹¹ is —OH, —O—Glc or —O—Glc—Glc; R¹² is H, —OH, —O—Glc, —O—Glc—Glc or —O—Glc—Rha; R¹³ is —OH or —O—Glc; R¹⁴ is —OH, —O—Glc, —O—Glc—Glc, or —O—Glc—Glc—Ac; and R¹⁵ is —H, —O—Glc, —O—Glc—Ac, or —O—Glc—Rha,
wherein Glc— is 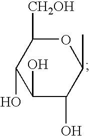
Glc—Glc— is 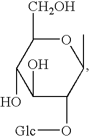
wherein Glc— is defined as above;
Rha—Glc— is 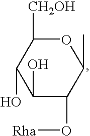
wherein Rha— is 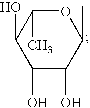
Ac—Glc— is 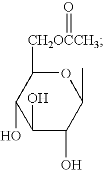
Ac—Glc—Glc— is 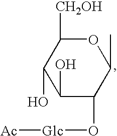
wherein Ac—Glc— is defined as above.

4. The dicarboxylic acid ester of ginsenoside of claim 3, wherein R⁴ and R⁹ are H, and n is 2 or 3.

5. The dicarboxylic acid ester of ginsenoside of claim 3, which is represented by the formula (I), wherein R^{12′} is H, and R^{11′} is —OH, and R^{13′} is —O—Glc′.

6. The dicarboxylic acid ester of ginsenoside of claim 3, which is represented by the formula (I), wherein R^{12′} is H, and R^{11′} is —O—Glc′, and R^{13′} is —OH.

7. The dicarboxylic acid ester of ginsenoside of claim 3, which is represented by the formula (I), wherein R^{12′} is H, and R^{11′} is —O—Glc—Glc′, and R^{13′} is —OH.

8. The dicarboxylic acid ester of ginsenoside of claim 3, which is represented by the formula (I), wherein R^{12′} is H, and R^{11′} is —O—Glc′, and R^{13′} is —O—Glc′.

9. The dicarboxylic acid ester of ginsenoside of claim 3, which is represented by the formula (I), wherein R^{12′} is —O—Glc′, and R^{11′} is —OH, and R^{13′} is —OH.

10. The dicarboxylic acid ester of ginsenoside of claim 3, which is represented by the formula (I), wherein R^{12′} is —O—Glc—Glc′, and R^{11 ′} is —OH, and R^{13′} is —OH.

11. The dicarboxylic acid ester of ginsenoside of claim 3, which is represented by the formula (I), wherein R^{12′} is —OH, and R^{11 ′} is —OH, and R^{13′} is —O—Glc′.

12. The dicarboxylic acid ester of ginsenoside of claim 3, which is represented by the formula (I), wherein R^{12′} is —O—Glc′, and R^{11′} is —OH, and R^{13′} is —O—Glc′.

13. The dicarboxylic acid ester of ginsenoside of claim 3, which is represented by the formula (I), wherein R^{12′} is —O—Glc—Rha′, and R^{11′} is —OH, and R^{13′} is —OH.

14. The dicarboxylic acid ester of ginsenoside of claim 3, which is represented by the formula (I), wherein R^{12′} is —H, and R^{11′} is —OH or —OE, and R^{13′} is —OH or —OE.

15. The dicarboxylic acid ester of ginsenoside of claim 3, which is represented by the formula (I), wherein R^{12′} is —OH or —OE, and R^{11′} is —OH or —OE, and R^{13′} is —OH or —OE.

16. The dicarboxylic acid ester of ginsenoside of claim 3, which is represented by the formula (II) or (III), wherein and R^{14′} and R^{15′} are one of the following combinations:

| R^{14′} | R^{15′} |
|---|---|
| —OH | —O-Glc-Rha′ |
| —O-Glc-Glc′ | H |
| —O-Glc′ | H |
| —OH | —O-Glc′ |
| —O-Glc-Glc-Ac′ | H |
| —OH | —O-Glc-Ac′ |

17. The dicarboxylic acid ester of ginsenoside of claim 3 having the formula (IV): 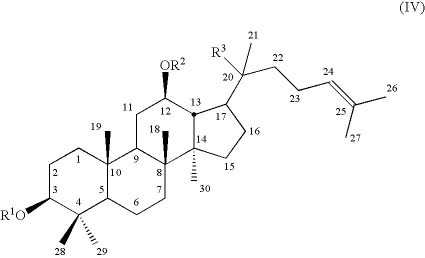
wherein
R¹=H or —C(O)(CR⁴R⁹)ₙCOOH;
R²=H or —C(O)(CR⁴R⁹)ₙCOOH;
R³= 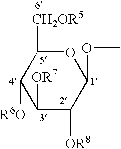
wherein R⁵, R⁶, R⁷ and R⁸ independently are H, or —(O)(CR⁴R⁹)ₙCOOH;
wherein n, R⁴ and R⁹ are defined as in claim 2.

18. The dicarboxylic acid ester of ginsenoside of claim 17, wherein R⁴ and R⁹ are H and n is 2 or 3.

19. The dicarboxylic acid ester of ginsenoside of claim 18, wherein R² is H.

20. The dicarboxylic acid ester of ginsenoside of claim 19, wherein R¹ is H.

21. The dicarboxylic acid ester of ginsenoside of claim 19, wherein R⁵is —C(O)(CH₂)ₙCOOH, and R⁶, R⁷ and R⁸ are all H.

22. The dicarboxylic acid ester of ginsenoside of claim 19, wherein R⁷is —C(O)(CH₂)ₙCOOH, and R⁵, R⁶ and R⁸ are all H.

23. The dicarboxylic acid ester of ginsenoside of claim 19, wherein R⁵ and R⁷ are both —C(O)(CH₂)ₙCOOH, and R⁶ and R⁸ are both H.

24. The dicarboxylic acid ester of ginsenoside of claim 19, wherein R⁵, R⁶ and R⁷ are all —C(O)(CH₂)ₙCOOH, and R⁸ is H.

25. The dicarboxylic acid ester of ginsenoside of claim 20, wherein R⁵is —C(O)(CH₂)ₙCOOH, and R⁶, R⁷ and R⁸ are all H.

26. The dicarboxylic acid ester of ginsenoside of claim 20, wherein R⁷is —C(O)(CH₂)ₙCOOH, and R⁵, R⁶ and R⁸ are all H.

27. The dicarboxylic acid ester of ginsenoside of claim 20, wherein R⁵ and R⁷ are both —C(O)(CH₂)ₙCOOH, and R⁶ and R⁸ are both H.

28. The dicarboxylic acid ester of ginsenoside of claim 20, wherein R⁵, R⁶ and R⁷ are all —C(O)(CH₂)ₙCOOH, and R⁸ is H.

29. A pharmaceutical preparation comprising an aqueous solution and a dicarboxylic acid ester of ginsenoside, or a pharmaceutically acceptable salt thereof, which is dissolved in said aqueous solution.

30. The pharmaceutical preparation of claim 29, wherein said dicarboxylic acid ester of ginsenoside comprises at least one group of —OC(O)—A—C(O)OH which replaces —OH of the ginsenoside, wherein A is —CR⁴R⁹)ₙ—, wherein R⁴ and R⁹ independently are H or C1-C4 alkyl, and n is an integer of 0-8.

31. The pharmaceutical preparation of claim 30, wherein said dicarboxylic acid ester of ginsenoside is represented by the following formulas (I), (II), (III-A) or (III-B): 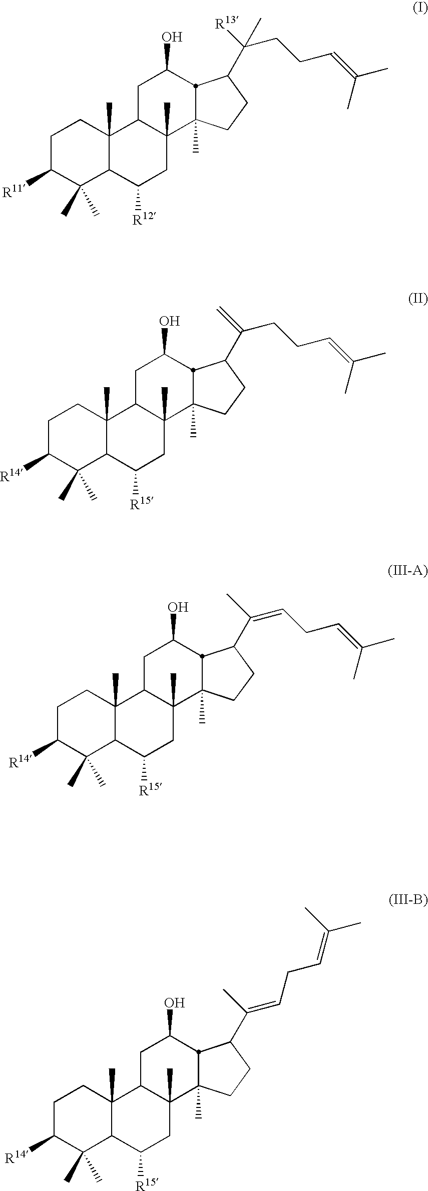
wherein R^{11′} is R¹¹, —OE, —O—Glc′ or —O—Glc—Glc′; R^{12′} is R¹², —OE, —O—Glc′, —O—Glc—Glc′ or —O—Glc—Rha′; R^{13′} is R₁₃, —OE or —O—Glc′; R^{14′} is R¹⁴, —OE, —O—Glc′, —O—Glc—Glc′, or —O—Glc—Glc—Ac′; and R^{15′} is R¹⁵, —O—Glc′, —O—Glc—Ac′, or —O—Glc—Rha′, wherein
E is —C(O)(CR⁴R⁹)ₙCOOH, wherein R⁴, R⁹ and n are defined as in claim 2;
—O—Glc′ is 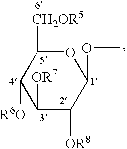
wherein R⁵, R⁶, R⁷ and R⁸ are H or E, wherein E is defined as above;
—O—Glc—Glc′ is 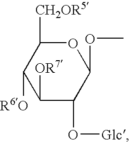
wherein R^{5′}, R^{6′}, and R^{7′} are H or E, wherein E and —O—Glc′ are defined as above;
—O—Glc—Rha′ is 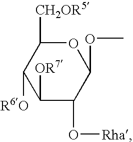
wherein R^{5′}, R^{6′}, and R^{7′} are H or E, wherein E is defined as above, and —O—Rha′ is 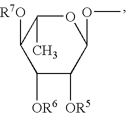
wherein R⁵, R⁶, and R⁷ are defined as above;
—O—Glc—Ac′ is 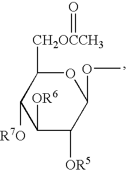
wherein R⁵,R⁶, and R⁷ are defined as above;
—O—Glc—Glc—Ac′ is 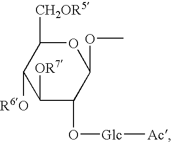
wherein R^{5′}, R^{6′}, R^{7′} and —O—Glc—Ac′ are defined as above;
wherein R¹¹ is —OH, —O—Glc or —O—Glc—Glc; R¹² is H, —OH, —O—Glc, —O—Glc—Glc or —O—Glc—Rha; R¹³ is —OH or —O—Glc; R¹⁴ is —OH, —O—Glc, —O—Glc—Glc, or —O—Glc—Glc—Ac; and R¹⁵ is —H, —O—Glc, —O—Glc—Ac, or —O—Glc—Rha,
wherein Glc— is 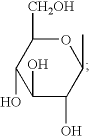
Glc—Glc— is 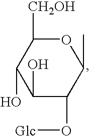
wherein Glc— is defined as above;
Rha—Glc— is 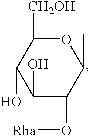
wherein Rha— is 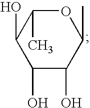
Ac—Glc— is 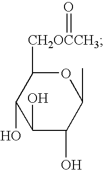
Ac—Glc—Glc— is 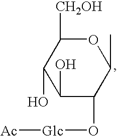
wherein Ac—Glc— is defined as above.

32. The pharmaceutical preparation of claim 31, wherein R⁴ and R⁹ are H, and n is 2 or 3.

33. The pharmaceutical preparation of claim 31, which is represented by the formula (I), wherein R^{12′} is H, and R^{11′} is —OH, and R^{13′} is —O—Glc′.

34. The pharmaceutical preparation of claim 31, which is represented by the formula (I), wherein R^{12′} is —OH, and R^{11′} is —OH, and Rh^{13′} is —O—Glc′.

35. The pharmaceutical preparation of claim 31, which is represented by the formula (I), wherein R^{12′} is —H, and R^{11′} is —OH or —OE, and R^{13′} is —OH or —OE.

36. The pharmaceutical preparation of claim 31, which is represented by the formula (I), wherein R^{12′} is —OH or —OE, and R^{11′} is —OH or —OE, and R^{13′} is —OH or —OE.

37. The pharmaceutical preparation of claim 31, wherein the concentration of said dicarboxylic acid ester of ginsenoside or a pharmaceutically acceptable salt thereof is 0.01-100 mg/ml in said aqueous solution.

38. The pharmaceutical preparation of claim 37, wherein the concentration of said dicarboxylic acid ester of ginsenoside or a pharmaceutically acceptable salt thereof is greater than 1 mg/ml in said aqueous solution.

39. The pharmaceutical preparation of claim 38, wherein the concentration of said dicarboxylic acid ester of ginsenoside or a pharmaceutically acceptable salt thereof is greater than 10 mg/ml in said aqueous solution.

40. The pharmaceutical preparation of claim 31 further comprising a pharmaceutically acceptable solubility enhancer of said dicarboxylic acid ester of ginsenoside.

41. The pharmaceutical preparation of claim 31, wherein said aqueous solution is a buffered or non-buffered aqueous solution.

42. A method for treating a tumor, said method comprising admninistering to a subject in need of treatment a dicarboxylic acid ester of ginsenoside, or a pharmaceutically acceptable salt thereof, in an amount effective to treat said tumor.

43. The method of claim 42, wherein said tumor is OVCAR-3, A549, HT-29 or MCF-7.

44. The method of claim 43, wherein said dicarboxylic acid ester of ginsenoside comprises at least one group of —OC(O)—A—C(O)OH which replaces —OH of the ginsenoside, wherein A is —(CR⁴R⁹)ₙ—, wherein R⁴ and R⁹ independently are H or C1-C4 alkyl, and n is an integer of 0-8.

45. The method of claim 44, wherein said dicarboxylic acid ester of ginsenoside is represented by the following formulas (I), (II), (III-A) or (III-B): 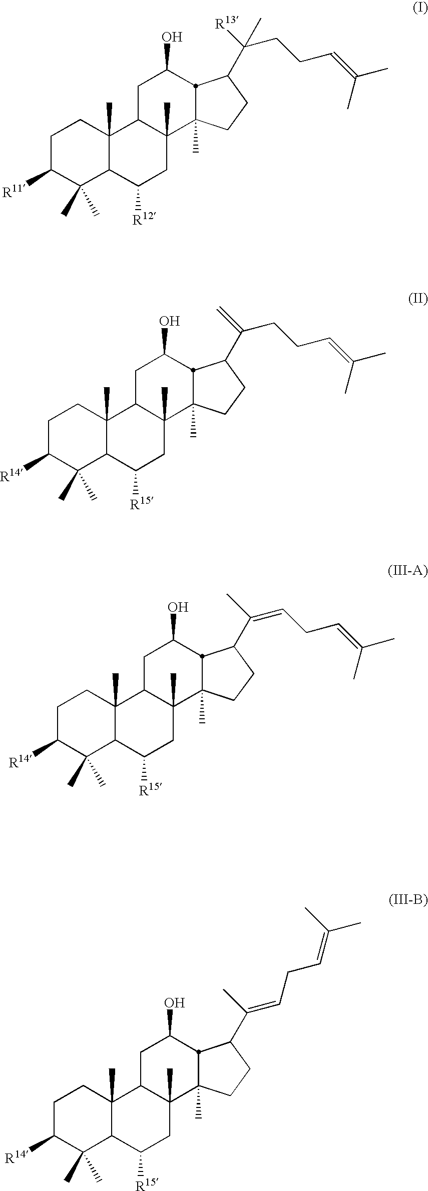
wherein R^{11′} is R¹¹, —OE, —O—Glc′ or —O—Glc—Glc′; R^{12′} is R¹², —OE, —O—Glc′, —O—Glc—Glc′ or —O—Glc—Rha′; R^{13′} is R₁₃, —OE or —O—Glc′; R^{14′} is R¹⁴, —OE, —O—Glc′, —O—Glc—Glc′, or —O—Glc—Glc—Ac′; and R^{15′} is R¹⁵, —O—Glc′, —O—Glc—Ac′, or —O—Glc—Rha′, wherein
E is —C(O)(CR⁴R⁹)ₙCOOH, wherein R⁴, R⁹ and n are defined as in claim 2;
—O—Glc′ is 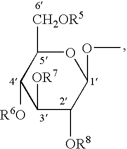
wherein R⁵, R⁶, R⁷ and R⁸ are H or E, wherein E is defined as above;
—O—Glc—Glc′ is 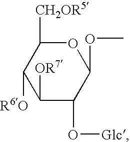
wherein R^{5′}, R^{6′}, and R^{7′} are H or E, wherein E and —O—Glc′ are defined as above;
—O—Glc—Rha′ is 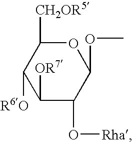
wherein R^{5′}, R^{6′}, and R^{7′} are H or E, wherein E is defined as above, and —O—Rha′ is 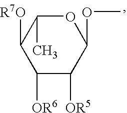
wherein R⁵, R⁶, and R⁷ are defined as above;
—O—Glc—Ac′ is 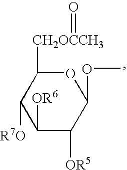
wherein R⁵,R⁶, and R⁷ are defined as above;
—O—Glc—Glc—Ac′ is 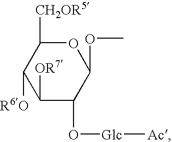
wherein R^{5′}, R^{6′}, R^{7′} and —O—Glc—Ac′ are defined as above;
wherein R¹¹ is —OH, —O—Glc or —O—Glc—Glc; R¹² is H, —OH, —O—Glc, —O—Glc—Glc or —O—Glc—Rha; R¹³ is —OH or —O—Glc; R¹⁴ is —OH, —O—Glc, —O—Glc—Glc, or —O—Glc—Glc—Ac; and R¹⁵ is —H, —O—Glc, —O—Glc—Ac, or —O—Glc—Rha,
wherein Glc— is 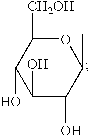
Glc—Glc— is 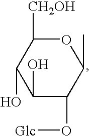
wherein Glc— is defined as above;
Rha—Glc— is 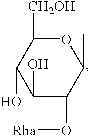
wherein Rha— is 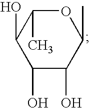
Ac—Glc— is 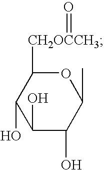
Ac—Glc—Glc— is 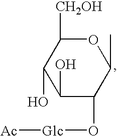
wherein Ac—Glc— is defined as above.

46. The method of claim 45, wherein R⁴ and R⁹ are H, and n is 2 or 3.

47. The method of claim 45, which is represented by the formula (I), wherein R^{12′} is H, and R^{11′} is —OH, and R^{13′} is —O—Glc′.

48. The method of claim 45, which is represented by the formula (I), wherein R^{12′} is —OH, and R^{11′} is —OH, and R^{13′} is —O—Glc′.

49. The method of claim 45, which is represented by the formula (I), wherein R^{12′} is —H, and R^{11 ′} is —OH or —OE, and R^{13′} is —H or —OE.

50. The method of claim 45, which is represented by the formula (I), wherein R^{12′} is —OH or —OE, and R^{11′} is —OH or —OE, and R^{13′} is —OH or —OE.

51. The method of claim 45, wherein said dicarboxylic acid ester of ginsenoside or a pharmaceutically acceptable salt is dissolved in an aqueous solution, and the concentration of said dicarboxylic acid ester of ginsenoside or a pharmaceutically acceptable salt thereof is 0.01-100 mg/ml in said aqueous solution.

52. The method of claim 51, wherein the concentration of said dicarboxylic acid ester of ginsenoside or a pharmaceutically acceptable salt thereof is greater than 1 mg/ml in said aqueous solution.

53. The method of claim 52, wherein the concentration of said dicarboxylic acid ester of ginsenoside or a pharmaceutically acceptable salt thereof is greater than 10 mg/ml in said aqueous solution.

54. The method of claim 51 wherein said aqueous solution further comprises a pharmaceutically acceptable solubility enhancer of said dicarboxylic acid ester of ginsenoside.

55. The method of claim 51, wherein said aqueous solution is a buffered or non-buffered aqueous solution.

56. A method for preparing a dicarboxylic acid ester of ginsenoside comprising reacting a ginsenoside with an activated dicarboxylic acid derivative in a solvent and in the presence of a catalyst to form a dicarboxylic acid ester of ginsenoside.

57. The method of claim 56, wherein said activated dicarboxylic acid derivative is dicarboxylic anhydride, dicarboxylic halide or dicarboxylic ester.

58. The method of claim 57, wherein said ginsenoside is represented by the following: 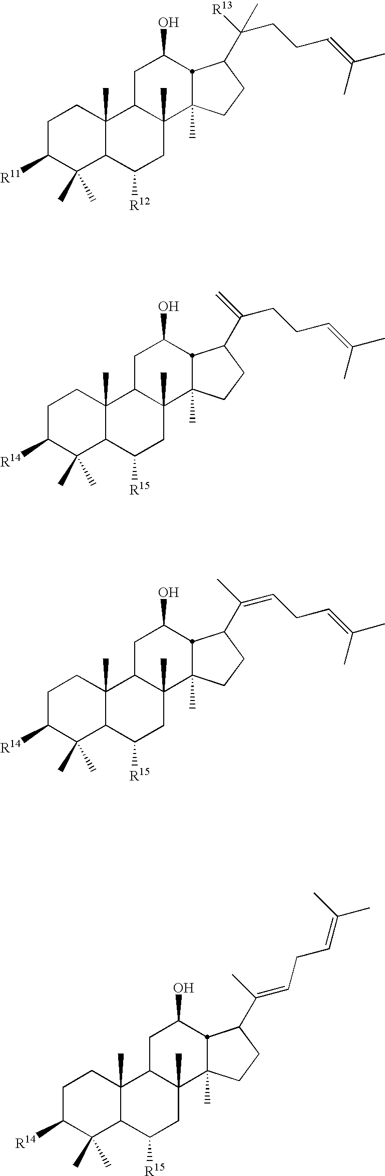
wherein R¹¹ is —OH, —O—Glc or —O—Glc-Glc; R¹² is H, —OH, —O—Glc, —O—Glc-Glc or —O—Glc—Rha; R¹³ is —OH or —O—Glc; R¹⁴ is —OH, —O—Glc, —O—Glc—Glc, or —O—Glc—Glc—Ac; and R¹⁵ is —H, —O—Glc, —O—Glc—Ac, or —O—Glc-Rha,
wherein Glc— is 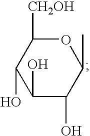
Glc—Glc— is 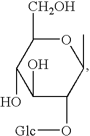
wherein Glc— is defined as above;
Rha—Glc— is 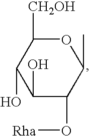
wherein Rha— is 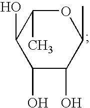
Ac—Glc— is 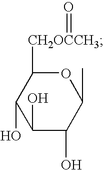
Ac—Glc—Glc— is 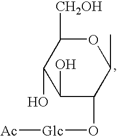
wherein Ac—Glc— is defined as above.

59. The method of claim 58, wherein said catalyst is a base, and said ginsenoside is 20-O-β-D-glucopyranosyl-protopanaxadiol having the following structure and said activated dicarboxylic acid derivative is a dicarboxylic acid anhydride having the following structure: 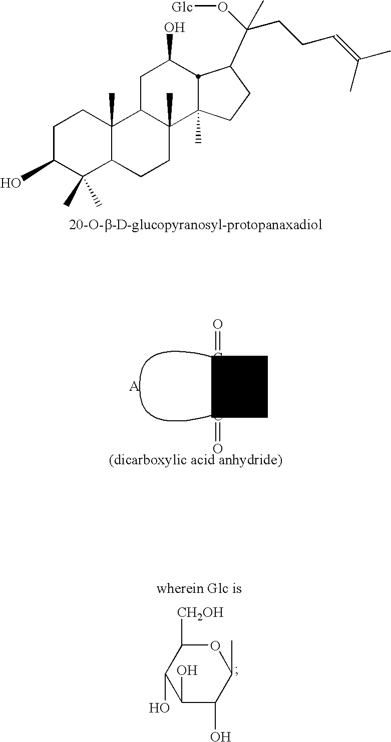
A is —(CR⁴R⁹)ₙ—; wherein R⁴ and R⁹ independently are H or C1-C4 alkyl, and n is an integer of 0-8.

60. The method of claim 59, wherein R⁴ and R⁹ are H, and n is 2 or 3.

61. The method of claim 59, wherein said solvent is halogenated alkane, ether, tertiary amine, low alkyl sulfoxide or low alkyl ketone.

62. The method of claim 61, wherein said halogenated alkane is dichloromethane, trichloromethane, or dichloroethane; said ether is dialkyl ether, alicyclic ether, tetrahydrofuran, or dioxane; said tertiary amine is triethylamine, pyridine, N,N,N′,N′-tetramethyl ethylenediamine, or N,N,N′,N′-tetramethyldiaminomethane; and said low alkyl sulfoxide is dimethyl sulfoxide.

63. The method of claim 59, wherein said base is solid alkali.

64. The method of claim 59, wherein said base is tertiary amine.

65. The method of claim 59, wherein said solvent and said base are tertiary amine.

66. The method of claim 65, wherein said tertiary amine is triethylamine, 4-(dimethylamino)pyridine, pyridine, N,N,N′,N′-tetramethyl ethylenediamine, or N,N,N′,N′-tetramethyldiaminomethane.
